(19) United States
(12) Patent Application Publication
(10) Pub. No.: US 2004/0030131 A1
(43) Pub. Date: Feb. 12, 2004

(54) Pharmaceutical compounds

(76) Inventors: Martine Keenan, Basingstoke (GB); Vincent Patrick Rocco, Indianapolis, IN (US); Kumiko Takeuchi, Indianapolis, IN (US); David Edward Tupper, Reading (GB); Vincent Vivien, Strasbourg (FR)

Correspondence Address: ELI LILLY AND COMPANY, PATENT DIVISION, P.O. BOX 6288, INDIANAPOLIS, IN, 46206-6288, US

(21) Appl. No.: 10/363,781

(22) PCT Filed: Sep. 21, 2001
(86) PCT No.: PCT/US01/27725

(30) Foreign Application Priority Data
Oct. 4, 2000 (GB) 0024318.8

(51) Int. Cl. C07D 413/02; C07D 417/02; C 07D 4 3/02

(52) U.S. Cl. 544/128; 544/135; 544/143; 544/145; 544/153; 544/363; 544/373; 544/376; 544/403; 546/152; 546/145; 546/198; 546/205; 548/950; 548/152; 548/465; 548/578

(57) ABSTRACT

A compound of the formula in which R1 and R2 are each hydrogen or C1-4 alkyl, or R1 and R2 together with the nitrogen atom to which they are attached form an azetidinyl, pyrrolidinyl, piperidinyl, piperazinyl or morpholino group, said group being optionally substituted with 1 to 3 substituents selected from C1-4 alkyl, hydroxymethyl, C1-4 alkoxymethyl and amido, R3 is a naphthyl, indolyl, benzothienyl, benzofuranyl, benzothiazolyl, quinolinyl or isoquinolinyl group, said group being optionally substituted, and n is 1 or 2; and m is 0 or 1, provided that when m is 1 then n is 1; or a salt or ester thereof.

This invention relates to novel compounds having pharmaceutical properties.

Certain aminoalkyl bicycloheptanes having a pharmacological effect on the central nervous system, are disclosed in British Patent 1 586 249. Also, British Patents 1 444 717 and 1 549 174 describe aminoalkyl bicyclooctyl derivatives with similar properties.

The compounds of the invention are of the following formula:

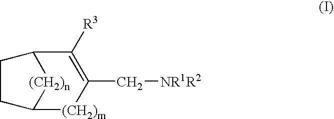

in which R¹ and R² are each hydrogen or C₁₋₄ alkyl, or R¹ and R² together with the nitrogen atom to which they are attached form an azetidinyl, pyrrolidinyl, piperidinyl, piperazinyl or morpholino group, said group being optionally substituted with 1 to 3 substituents selected from C₁₋₄ alkyl, hydroxymethyl,

C₁₋₄ alkoxymethyl and amido,

R³ is a naphthyl, indolyl, benzothienyl, benzofuranyl, benzothiazolyl, quinolinyl or isoquinolinyl group, said group being optionally substituted, and

n is 1 or 2 and m is 0 or 1, provided that when m is 1 then n is 1;

or a salt or ester thereof.

The compounds of the invention and their pharmaceutically acceptable salts and esters are indicated for use in the treatment of disorders of the central nervous system.

In the above formula (I), R¹ and R² are preferably hydrogen or C₁₋₄ alkyl. When R¹ and R² combine to form one of the cyclic rings mentioned above, they can be substituted with C₁₋₄ alkyl, hydroxymethyl, C₁₋₄ alkoxymethyl or amido. An amido group is preferably a C2-5 acylamido substituent of the formula C₁₋₄ alkyl CONH—, and is most preferebly acetamido.

A C₁₋₄ alkyl group can be methyl, ethyl, propyl or butyl, and can be branched or unbranched and includes isopropyl and tert-butyl. Preferably R¹ and R² are each hydrogen, methyl or ethyl, and especially hydrogen or methyl. The —NR¹R² group is preferably —N(CH₃)₂ and most preferably —NH(CH₃).

A C₁₋₄ alkoxy is an alkyl group linked to an oxygen atom, where the alkyl is as defined above. A C₁₋₄ alkoxy group includes for example methoxy and ethoxy.

Compounds of formula (I) in which n is 1 and m is 0, are 2.2.1 bicycloheptyl derivatives of the structure:

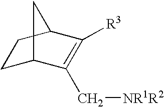

Compounds of formula (I) in which n is 1 and m is 1, are 3.2.1 bicyclooctyl derivatives of the structure

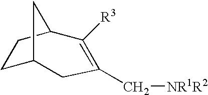

Compounds of formula (I) in which n is 2 and m is 0 are 2.2.2 bicycloocyl derivatives of the structure:

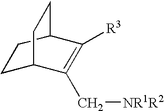

The R³ substituent is attached to the bicyclo ring at certain positions on the substituent, and examples of R³ groups are α-naphthyl, β-naphthyl, 2-, 3-, 4-, 5-, 6- or 7-indolyl, 2-, 3-, 4-, 5-, 6- or 7-benzothienyl, 2-, 3-, 4-, 5-, 6- or 7-benzofuranyl, 2-, 3-, 4-, 5-, 6- or 7-benzothiazolyl, 2-, 3-, 4-, 5-, 6- or 7-quinolinyl or 3-,4-, 5-, 6- or 7-isoquinolinyl. Preferred R³ substituents are, 2-, 3-, 4-, 5-, 6- or 7-benzothienyl, and especially 4-, 5- or 7-benzothienyl.

The R³ group can also be substituted, substitution being in one or both rings, with one or more, preferably 1 to 3, substituents. Preferred substituents include C₁₋₄ alkyl, C₁₋₄ alkoxy, carboxy, nitro, hydroxy, cyano, halo, trifluoromethyl, trifluoromethoxy, optionally substituted benzyl, optionally substituted benzyloxy, —NR′R″, —CONR′R″, —SO₂NR′R″ and —SO₂R′, where R′ and R″ are each hydrogen or C₁₋₄ alkyl. Especially preferred substituents are C₁₋₄ alkyl, halo and cyano. A halo substituent is preferably chloro, bromo or fluoro. A benzyl or benzyloxy group is optionally substituted on the phenyl ring with one of the substituents listed above or with 1 to 3 of the substituents listed above, i.e., selected from C₁₋₄ alkyl, C₁₋₄ alkoxy, carboxy, nitro, hydroxy, cyano, halo, trifluoromethyl, trifluoromethoxy, benzyl, benzyloxy —NR′R″, —CONR′R″, —SO₂NR′R″ and —SO₂R′, where R′ and R″ are as defined above.

As indicated above, it is possible to prepare salts of the compound of the invention and such salts are included in the invention. Such salts are preferably the pharmaceutically acceptable, non-toxic salts. Of special interest are acid addition salts, in particular those with suitable acids, such as those with inorganic acids, for example hydrochloric, hydrobromic, nitric, sulphuric or phosphoric acids, or with organic acids, such as organic carboxylic acids, for example, maleic, fumaric, tartaric or citric acid.

In addition to the pharmaceutically acceptable salts, other salts are included in the invention. They may serve as intermediates in the purification of compounds or in the preparation of other, for example pharmaceutically acceptable, acid addition salts, or are useful for identification, characterisation or purification.

It will be appreciated that when a substituent on an R³ group is acidic such as, for example, a carboxy group, the opportunity exists for esters. These can be aliphatic or aromatic, being preferably alkyl esters derived from C₁₋₄ alkanols, especially methyl and ethyl esters. An example of an ester substituent is —COOR′ where R′ is C₁₋₄ alkyl.

The bicyclo compounds of the 2.2.1 and 3.2.1 systems contain asymmetric carbon atoms as indicated by asterisks in the following structures:

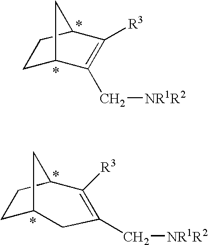

Thus R and S enantiomeric forms of these compounds exist. The compounds can be prepared as racemic mixtures and can conveniently be used a such, but individual isomers can be isolated by conventional techniques, or are preferably prepared by chiro-selective methods. Both racemic mixtures and individual isomers are included in the present invention. A preferred group of compounds of formula (I) is one in which R¹ and R² are each hydrogen or C₁₋₄ alkyl, and R³ is optionally substituted benzothienyl, said optionally substituted benzothienyl preferably being attached at the 4- or 7-position.

A further preferred group of compounds of the invention is one in which R³ is an optionally substituted benzothienyl group attached at 5-position and preferably wherein said benzothienyl is substituted in the 3-position with Halogen or C₁₋₄ alkyl, preferably methyl.

An even further preferred group of compounds of the invention can be represented as follows:

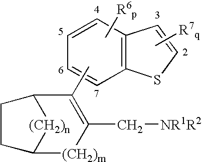

in which R¹ and R² are each hydrogen or C₁₋₄ alkyl, R⁶ and R⁷ are each C₁₋₄ alkyl, C₁₋₄ alkoxy, carboxy, hydroxy, cyano, halo, trifluoromethyl, —NR′R″, —CONR′R″, —SO₂NR′R″ or —SO₂R′ where R′ and R″ are each hydrogen or C₁:₄ alkyl, and p and q are each 0, 1 or 2; or a salt thereof.

The bicyclo moiety can be attached at the 4-, 5-, 6- or 7-position, preferably the 4- or 7-position, and most preferably at the 4-position.

Examples of compounds of the invention, which can be as free base or in the form of a pharmaceutically acceptable salt, and in isomeric or racemic forms, are as follows:

dimethyl-[3-(4-methoxybenzo[b]thiophen-2-yl)bicyclo[2.2.1]hept-2-en-2-yl]methanamine,

N,N-dimethyl [3-(2-naphthyl)bicyclo[2.2.1]hept-2-en-2-yl]methanamine,

[3-(1-benzothien-2-yl)bicyclo[2.2.1]hept-2-en-2-yl]-N,N-dimethylmethanamine,

[3-(3-bromo-1-benzothien-2-yl)bicyclo[2.2.1]hept-2-en-2-yl]-N,N-dimethylmethanamine,

[3-(6-methoxy-2-naphthyl)bicyclo[2.2.1]hept-2-en-2-yl]-N,N-dimethylmethanamine,

6-(3-[(dimethylamino)methyl]bicyclo[2.2.1]hept-2-en-2-yl}-2-naphthol,

[3-(6-bromo-2-naphthyl)bicyclo[2.2.1]hept-2-en-2-yl]-N,N-dimethylmethanamine,

[3-(1-benzofuran-2-yl)bicyclo[2.2.1]hept-2-en-2-yl]-N,N-dimethylmethanamine,

[3-(6-fluoro-2-naphthyl)bicyclo[2.2.1]hept-2-en-2-yl]-N,N-dimethylmethanamine,

(1-benzothien-5-yl)bicyclo[2.2.1]hept-2-en-2-yl]-N,N-dimethylmethanamine,

[3-(1-benzothien-3-yl)bicyclo[2.2.1]hept-2-en-2-yl]-N,N-dimethylmethanamine,

N,N-dimethyl[3-(3-quinolinyl)bicyclo[2.2.1]hept-2-en-2-yl]methanamine,

N,N-dimethyl[3-(5-methyl-1-benzothien-2-yl)bicyclo[2.2.1]hept-2-en-2-yl]methanamine,

N,N-dimethyl[3-(6-methyl-2-naphthyl)bicyclo[2.2.1]hept-2-en-2-yl]methanamine,

[3-(1-benzothien-6-yl)bicyclo[2.2.1]hept-2-en-2-yl]-N,N-dimethylmethanamine,

[3-(6-chloro-1,3-benzothiazol-2-yl)bicyclo[2.2.1]hept-2-en-2-yl]-N,N-dimethylmethanamine,

N,N-dimethyl{3-[6-(methylsulfonyl)-2-naphthyl]bicyclo[2.2.1]hept-2-en-2-yl}methanamine,

[3-(6-methoxy-1-benzothien-2-yl)bicyclo[2.2.1]hept-2-en-2-yl]-N,N-dimethylmethanamine,

[3-(5-methoxy-1-benzothien-2-yl)bicyclo[2.2.1]hept-2-en-2-yl]-N,N-dimethylmethanamine,

[3-(8-methoxy-2-naphthyl)bicyclo[2.2.1]hept-2-en-2-yl]-N,N-dimethylmethanamine,

N,N-dimethyl[3-(2-quinolinyl)bicyclo[2.2.1]hept-2-en-2-yl]methanamine,

[3-(4-isoquinolinyl)bicyclo[2.2.1]hept-2-en-2-yl]-N,N-dimethylmethanamine,

N,N-dimethyl[3-(6-quinolinyl)bicyclo[2.2.1]hept-2-en-2-yl]methanamine,

N,N-dimethyl[3-(2-quinolinyl)bicyclo[2.2.1]hept-2-en-2-yl]methanamine,

[3-(4-isoquinolinyl)bicyclo[2.2.1]hept-2-en-2-yl]-N,N-dimethylmethanamine,

N,N-dimethyl[3-(8-quinolinyl)bicyclo[2.2.1]hept-2-en-2-yl]methanamine,

N,N-dimethyl[3-(5-quinolinyl)bicyclo[2.2.1]hept-2-en-2-yl]methanamine,

[3-(6-fluoro-1-benzothien-3-yl)bicyclo[2.2.1]hept-2-en-2-yl]-N,N-dimethylmethanamine,

N,N-dimethyl [3-(2-methyl-6-quinolinyl)bicyclo[2.2.1]hept-2-en-2-yl]methanamine,

N,N-dimethyl[3-(4-methyl-2-quinolinyl)bicyclo[2.2.1]hept-2-en-2-yl]methanamine,

N,N-dimethyl[3-(2-methyl-1,3-benzothiazol-5-yl)bicyclo[2.2.1]hept-2-en-2-yl]methanamine,

[3-(5-methoxy-2-naphthyl)bicyclo[2.2.1]hept-2-en-2-yl]-N,N-dimethylmethanamine,

[3-(6-fluoro-1,3-benzothiazol-2-yl)bicyclo[2.2.1]hept-2-en-2-yl]-N,N-dimethylmethanamine,

[3-(4-chloro-1,3-benzothiazol-2-yl)bicyclo[2.2.1]hept-2-en-2-yl]-N,N-dimethylmethanamine,

[3-(6-methoxy-1,3-benzothiazol-2-yl)bicyclo[2.2.1]hept-2-en-2-yl]-N,N-dimethylmethanamine,

N,N-dimethyl [3-(1-naphthyl)bicyclo[2.2.1)hept-2-en-2-yl]methanamine,

[3-(6-chloro-1-benzothien-2-yl)bicyclo[2.2.1]hept-2-en-2-yl]-N,N-dimethylmethanamine,

[3-(4-methoxy-1,3-benzothiazol-2-yl)bicyclo[2.2.1]hept-2-en-2-yl]-N,N-dimethylmethanamine,

[3-(7-bromo-4-methoxy-1,3-benzothiazol-2-yl)bicyclo[2.2.1]hept-2-en-2-yl]-N,N-dimethylmethanamine,

N,N-dimethyl{3-[6-(trifluoromethyl)-1-benzothien-2-yl]bicyclo[2.2.1]hept-2-en-2-yl}methanamine,

[3-(6-chloro-1-benzothien-3-yl)bicyclo[2.2.1]hept-2-en-2-yl]-N,N-dimethylmethanamine,

N,N-dimethyl{3-[6-(trifluoromethyl)-1-benzothien-3-yl]bicyclo[2.2.1]hept-2-en-2-yl}methanamine,

[3-(4-fluoro-1-naphthyl)bicyclo[2.2.1]hept-2-en-2-yl]-N,N-dimethylmethanamine,

N,N-dimethyl[3-(6-methyl-1-benzothien-2-yl)bicyclo[2.2.1]hept-2-en-2-yl]methanamine,

1-{[3-(2-naphthyl)bicyclo[2.2.1]hept-2-en-2-yl]methyl}piperidine,

4-{[3-(2-naphthyl)bicyclo[2.2.1]hept-2-en-2-yl]methyl}morpholine,

1-methyl-4-{[3-(2-naphthyl)bicyclo[2.2.1]hept-2-en-2-yl]methyl}piperazine,

1-{[3-(2-naphthyl)bicyclo[2.2.1]hept-2-en-2-yl]methyl}piperazine

N-ethyl-N-{[3-(2-naphthyl)bicyclo[2.2.1]hept-2-en-2-yl]methyl}ethanamine,

4-(6-methoxy-2-naphthyl)-2-methyl-1-{[3-(2-naphthyl)bicyclo[2.2.1]hept-2-en-2-yl]methyl}piperidine

2-methyl-1-{[3-(2-naphthyl)bicyclo[2.2.1]hept-2-en-2-yl]methyl}pyrrolidine,

methyl (-1-{[3-(2-naphthyl)bicyclo[2.2.1]hept-2-en-2-yl]methyl}-2-pyrrolidinyl)methyl ether,

(-1-{[3-(2-naphthyl)bicyclo[2.2.1]hept-2-en-2-yl]methyl}-2-pyrrolidinyl)methanol,

N-(2,5-Dimethyl)pyrrolidyl-[2-(2-naphthyl)bicyclo[2.2.1]-2-hepten-3-yl]methylamine,

N-(2R-Hydroxymethyl)pyrrolidyl-[2-(2-naphthyl)bicyclo [2.2.1]-2-hepten-3-yl]methylamine,

N-(-Hydroxytethyl)pyrrolidyl-[2-(2-naphthyl)bicyclo [2.2.11-2-hepten-3-yl]methylamine,

1-{[3-(2-naphthyl)bicyclo[2.2.1]hept-2-en-2-yl]methyl}-3-pyrrolidinol,

N-(1-{[3-(2-naphthyl)bicyclo[2.2.1]hept-2-en-2-yl]methyl}-3-pyrrolidinyl)acetamide,

1-{[3-(6-fluoro-1-benzothien-3-yl)bicyclo[2.2.1]hept-2-en-2-yl]methyl}pyrrolidine,

1-{[3-(3-methyl-1-benzothien-5-yl)bicyclo[2.2.1]hept-2-en-2-yl]methyl}pyrrolidine,

2,2-dimethyl-1-{[3-(2-naphthyl)bicyclo[2.2.1]hept-2-en-2-yl]methyl}pyrrolidine,

N-{[3-(1-benzothien-7-yl)bicyclo[2.2.1]hept-2-en-2-yl]methyl}-N-methyl-2-propen-1-amine,

[3-(1-benzothien-7-yl)bicyclo[2.2.1]hept-2-en-2-yl]-N-methylmethanamine,

N,N-Pyrrolidyl-[2-(2-naphthyl)bicyclo[2.2.11-2-hepten-3-yl]methanamine,

dimethyl-[3-(5-fluorobenzo[b]thiophen-2-yl)bicyclo [2.2.1]hept-2-en-2-yl]methanamine,

dimethyl-[3-(4-fluorobenzo[b]thiophen-2-yl)bicyclo[2.2.1]hept-2-en-2-yl]methanamine,

dimethyl-[3-(6-fluorobenzo[b]thiophen-2-yl)bicyclo[2.2.1]hept-2-en-2-yl]methanamine,

dimethyl-[3-(7-fluorobenzo[b]thiophen-2-yl)bicyclo[2.2.1]hept-2-en-2-yl]methanamine,

dimethyl-[3-(7-methoxybenzo[b]thiophen-2-yl)bicyclo[2.2.1]hept-2-en-2-yl]methanamine,

(3-(6-fluorobenzo[b]thiophen-2-yl)bicyclo[2.2.1]hept-2-en-2-yl)methyl) methanamine,

[3-(1,3-benzothiazol-2-yl)bicyclo[2.2.1]hept-2-en-2-yl]-N-methylmethanamine,

[3-(6-chloro-1,3-benzothiazol-2-yl)bicyclo[2.2.1]hept-2-en-2-yl]-N-methylmethanamine,

N-benzyl-N-{[3-(6-methoxy-2-naphthyl)bicyclo[2.2.1]hept-2-en-2-yl]methyl}-N-methanamine,

((3-(benzo[b]thiophen-4-yl)bicyclo[2.2.1]-hept-2-en-2-yl)methyl)dimethanamine,

dimethyl(2-(5-fluorobenzo[b]thiophen-2-yl)bicyclo[3.2.1]-oct-2-en-3-yl) methanamine,

N,N-dimethyl[2-(2-naphthyl)bicyclo[3.2.1]oct-2-en-3-yl]methanamine,

N,N-dimethyl[2-(6-fluoro-2-naphthyl)bicyclo[3.2.1]oct-2-en-3-yl]methanamine,

N,N-dimethyl[2-(6-fluoro-2-naphthyl)bicyclo [3.2.1]oct-2-en-3-yl]methanamine,

[2-(7-fluoro-1-benzothien-2-yl)bicyclo[3.2.1]oct-2-en-3-yl]-N,N-dimethylmethanamine,

[2-(4-fluoro-1-benzothien-2-yl)bicyclo[3.2.1]oct-2-en-3-yl]-N,N-dimethylmethanamine,

[2-(6-fluoro-1-benzothien-2-yl)bicyclo[3.2.1]oct-2-en-3-yl]-N,N-dimethylmethanamine,

[2-(6-fluoro-1-benzothien-2-yl)bicyclo [3.2.1]oct-2-en-3-yl]-N,N-dimethylmethanamine,

[2-(6-fluoro-1-benzothien-3-yl)bicyclo [3.2.1]oct-2-en-3-yl]-N,N-dimethylmethanamine,

2-{3-[(dimethylamino)methyl]bicyclo[3.2.1]oct-2-en-2-yl}-1-benzothiophene-6-carbonitrile,

[2-(6-trifluoro-1-benzothien-3-yl)bicyclo[3.2.1]oct-2-en-3-yl]-N,N-dimethylmethanamine,

N,N-dimethyl[3-(2-naphthyl)bicyclo[2.2.2]oct-2-en-2-yl]methanamine,

[3-(6-methoxy-2-naphthyl)bicyclo[2.2.2]oct-2-en-2-yl]-N,N-dimethylmethanamine,

[3-(1-benzothien-3-yl)bicyclo[2.2.2]oct-2-en-2-yl]-N,N-dimethylmethanamine,

6-{3-[(dimethylamino)methyl]bicyclo[2.2.2]oct-2-en-2-yl}-2-naphthol,

[3-(6-fluoro-2-naphthyl)bicyclo[2.2.2]oct-2-en-2-yl]-N,N-dimethylmethanamine,

[3-(6-fluoro-1-benzothien-3-yl)bicyclo[2.2.2]oct-2-en-2-yl]-N,N-dimethylmethanamine,

1-{[3-(1-benzothien-5-yl)bicyclo[3.2.1]oct-2-en-2-yl]methyl}pyrrolidine,

1-{[3-(1-benzothien-5-yl)bicyclo[3.2.1]oct-2-en-2-yl]methyl}azetidine,

1-{[3-(3-methyl-1-benzothien-5-yl)bicyclo[2.2.2]oct-2-en-2-yl]methyl}pyrrolidine,

[3-(1-benzothien-7-yl)bicyclo[2.2.2]oct-2-en-2-yl]-N-methylmethanamine,

[2-(1-benzothien-5-yl)bicyclo[3.2.1]oct-2-en-3-yl]-N,N-dimethylmethanamine,

2-(1-benzothien-5-yl)bicyclo[3.2.1]oct-2-en-3-yl]-N,N-dimethylmethanamine,

2-(1-benzothien-5-yl)bicyclo[3.2.1]oct-2-en-3-yl]-N,N-dimethylmethanamine,

[2-(1-benzothien-6-yl)bicyclo[3.2.1]oct-2-en-3-yl]-N,N-dimethylmethanamine,

[2-(1-benzothien-4-yl)bicyclo[3.2.1]oct-2-en-3-yl]-N,N-dimethylmethanamine,

[2-(1-benzothien-4-yl)bicyclo[3.2.1]oct-2-en-3-yl]-N,N-dimethylmethanamine,

[2-(1-benzothien-7-yl)bicyclo[3.2.1]oct-2-en-3-yl]-N,N-dimethylmethanamine,

[2-(1-benzothien-7-yl)bicyclo[3.2.1]oct-2-en-3-yl]-N,N-dimethylmethanamine,

[2-(3-chloro-1-benzothien-5-yl)bicyclo[3.2.1)oct-2-en-3-yl]-N,N-dimethylmethanamine,

N,N-dimethyl[2-(3-methyl-1-benzothien-5-yl)bicyclo [3.2.1]oct-2-en-3-yl]methanamine,

N,N-dimethyl[2-(3-methyl-1-benzothien-5-yl)bicyclo [3.2.1]oct-2-en-3-yl]methanamine,

N,N-dimethyl[2-(3-methyl-1-benzothien-7-yl)bicyclo [3.2.1]oct-2-en-3-yl]methanamine,

[3-(1-benzothien-5-yl)bicyclo[2.2.2]oct-2-en-2-yl]-N,N-dimethylmethanamine,

[3-(1-benzothien-6-yl)bicyclo[2.2.2]oct-2-en-2-yl]-N,N-dimethylmethanamine,

[3-(3-chloro-1-benzothien-5-yl)bicyclo[2.2.2]oct-2-en-2-yl]-N,N-dimethylmethanamine,

N,N-dimethyl[3-(3-methyl-1-benzothien-5-yl)bicyclo [2.2.2]oct-2-en-2-yl]methanamine,

[3-(1-benzothien-7-yl)bicyclo[2.2.2]oct-2-en-2-yl]-N,N-dimethylmethanamine,

[3-(1-benzothien-4-yl)bicyclo[2.2.2]oct-2-en-2-yl]-N,N-dimethylmethanamine,

N-methyl-N-{[2-(3-methyl-1-benzothien-5-yl) bicyclo [3.2.1]oct-2-en-3-yl]methanamine,

[3-(6-fluoro-2-naphthyl)bicyclo[3.2.1]oct-2-en-2-yl]-N-methylmethanamine,

N,N-dimethyl[3-(6-{[4-(trifluoromethyl)benzyl]oxy}-2-naphthyl)bicyclo[2.2.2]oct-2-en-2-yl]methanamine,

[3-(6-methoxy-2-naphthyl)bicyclo[2.2.2]oct-2-en-2-yl]-N,N-dimethylmethanamine,

[3-(6-butoxy-2-naphthyl)bicyclo[2.2.2]oct-2-en-2-yl]-N,N-dimethylmethanamine,

{3-[6-(benzyloxy)-2-naphthyl]bicyclo[2.2.2]oct-2-en-2-yl}-N,N-dimethylmethanamine,

(3-{6-[(4-bromobenzyl)oxy]-2-naphthyl}bicyclo[2.2.2]oct-2-en-2-yl)-N,N-dimethylmethanamine,

4-{[(6-{3-[(dimethylamino)methyl]bicyclo[2.2.2]oct-2-en-2-yl}-2-naphthyl)oxy]methyl}benzonitrile,

(3-[(6-[(3-chlorobenzyl)oxy]-2-naphthyl}bicyclo[2.2.2]oct-2-en-2-yl)-N,N-dimethylmethanamine,

N,N-dimethyl(3-{6-[(4-methylbenzyl)oxy]-2-naphthyl}bicyclo[2.2.2]oct-2-en-2-yl)methanamine,

(3-{6-[(2,6-dichlorobenzyl)oxy]-2-naphthyl}bicyclo [2.2.2]oct-2-en-2-yl)-N,N-dimethylmethanamine,

[3-(6-ethoxy-2-naphthyl)bicyclo[2.2.2]oct-2-en-2-yl]-N,N-dimethylmethanamine,

(3-(6-[(4-tert-butylbenzyl)oxy]-2-naphthyl)bicyclo [2.2.2]oct-2-en-2-yl)-N,N-dimethylmethanamine,

(3-{6-[(2-chlorobenzyl)oxy]-2-naphthyl}bicyclo [2.2.2]oct-2-en-2-yl)-N,N-dimethylmethanamine,

(3-{6-[(4-fluorobenzyl)oxy]-2-naphthyl}bicyclo [2.2.2]oct-2-en-2-yl)-N,N-dimethylmethanamine,

N,N-dimethyl[3-(6-propoxy-2-naphthyl)bicyclo[2.2.2]oct-2-en-2-yl]methanamine,

(3-{6-[(4-methoxybenzyl)oxy]-2-naphthyl}bicyclo [2.2.2]oct-2-en-2-yl)-N,N-dimethylmethanamine,

N,N-dimethyl(3-{6-[(4-nitrobenzyl)oxy]-2-naphthyl}bicyclo[2.2.2]oct-2-en-2-yl)methanamine,

N,N-dimethyl[3-{6-{[4-(trifluoromethoxy)benzyl]oxy}-2-naphthyl)bicyclo[2.2.2]oct-2-en-2-yl]methanamine,

[(1R,4S)-N,N-dimethyl-3-(1-benzothien-4-yl)bicyclo [2.2.1]hept-2-en-2-yl]-methanamine hydrochloride,

[(1S,4R)-N,N-dimethyl-3-(1-benzothien-4-yl)bicyclo [2.2.1]hept-2-en-2-yl]-methanamine hydrochloride,

[N-methyl-(3-(3-methyl-1-benzothien-7-yl)bicyclo [2.2.1]hept-2-en-2-yl]-methanamine hydrochloride,

[(1R,4S)-N,N-dimethyl-3-(1-benzothien-7-yl)bicyclo [2.2.1]hept-2-en-2-yl]-methanamine hydrochloride,

[(1S,4R)-N,N-dimethyl-3-(1-benzothien-7-yl)bicyclo [2.2.1]hept-2-en-2-yl]-methanamine hydrochloride,

[N,N-dimethyl 3-(2-methoxymethyl-1-benzothien-6-yl)bicyclo[2.2.1]hept-2-en-2-yl]-methanamine hydrochloride,

[N,N-dimethyl 3-(2-hydroxymethyl-1-benzothien-6-yl)bicyclo[2.2.1]hept-2-en-2-yl]-methanamine hydrochloride,

[N,N-dimethyl 3-(2-{prop-2-enyl}-1-benzothien-6-yl)bicyclo[2.2.1]hept-2-en-2-yl]-methanamine hydrochloride,

[N-methyl-3-(1-benzothien-4-yl)bicyclo[2.2.1]hept-2-en-2-yl]-methanamine hydrochloride,

[N-methyl-3-(6-fluoro-1-benzothien-3-yl)bicyclo [2.2.1]hept-2-en-2-yl]-methanamine hydrochloride,

[(1R,4S)-N-methyl-3-(1-benzothien-7-yl)bicyclo [2.2.1]hept-2-en-2-yl]-methanamine hydrochloride,

[(1S,4R)-N-methyl-3-(1-benzothien-7-yl)bicyclo [2.2.1]hept-2-en-2-yl]-methanamine hydrochloride,

[(1R,4S)-N,N-dimethyl-3-(3-methyl-1-benzothien-7-yl)bicyclo[2.2.1]hept-2-en-2-yl]-methanamine hydrochloride,

[(1S,4R)-N,N-dimethyl-3-(3-methyl-1-benzothien-7-yl)bicyclo[2.2.1]hept-2-en-2-yl]-methanamine hydrochloride,

[(1R,4S)-N,N-dimethyl-3-(1-benzothien-2-yl)bicyclo [2.2.1]hept-2-en-2-yl]-methanamine hydrochloride,

[(1S,4R)-N,N-dimethyl-3-(1-benzothien-2-yl)bicyclo [2.2.1]hept-2-en-2-yl]-methanamine hydrochloride,

[(1R,4S)-N-methyl-(3-(3-methyl-1-benzothien-7-yl)bicyclo [2.2.1]hept-2-en-2-yl]-methanamine hydrochloride,

[(1S,4R)-N-methyl-(3-(3-methyl-1-benzothien-7-yl)bicyclo [2.2.1]hept-2-en-2-yl]-methanamine hydrochloride,

[N-methyl-(3-(3-methyl-1-benzothien-6-yl)bicyclo [2.2.1]hept-2-en-2-yl]-methanamine hydrochloride,

[(1R,4S)-N-methyl-(3-(1-benzothien-7-yl)bicyclo [2.2.1]hept-2-en-2-yl]-methanamine hydrochloride,

[(1S,4R)-N-methyl-(3-(1-benzothien-7-yl)bicyclo [2.2.1]hept-2-en-2-yl]-methanamine hydrochloride,

[N-methyl-(3-(5-fluoro-1-benzothien-7-yl)bicyclo [2.2.1]hept-2-en-2-yl]-methanamine hydrochloride,

[N-methyl-(3-(3-cyano-1-benzothien-5-yl)bicyclo [2.2.1]hept-2-en-2-yl]-methanamine hydrochloride,

[N-methyl-(3-(1-benzofuran-5-yl)bicyclo[2.2.1]hept-2-en-2-yl]-methanamine hydrochloride,

[(1R,4S)-N-methyl-3-(6-fluoro-1-benzothien-3-yl)bicyclo [2.2.1]hept-2-en-2-yl]-methanamine hydrochloride,

[(1S,4R)-N-methyl-3-(6-fluoro-1-benzothien-3-yl)bicyclo [2.2.1]hept-2-en-2-yl]-methanamine hydrochloride,

N-allyl-N-methyl-[3-(1-benzothien-5-yl)bicyclo [2.2.2]oct-2-en-2-yl]-methanamine hydrochloride,

N,N-dimethyl-[3-(3-methyl-1-benzothien-7-yl)bicyclo [2.2.2] oct-2-en-2-yl]-methanamine hydrochloride,

N,N-dimethyl-[3-(1-benzothien-2-yl)bicyclo[2.2.2]oct-2-en-2-yl]-methanamine hydrochloride,

N,N-dimethyl-[(1S,5R)-2-(3-chloro-1-benzothien-5-yl)bicyclo[3.2.1]oct-2-en-3-yl]methanamine hydrochloride,

N,N-dimethyl-[(1R,5S)-2-(3-chloro-1-benzothien-5-yl)bicyclo[3.2.1]oct-2-en-3-yl]methanamine hydrochloride,

N,N-dimethyl-[(1S,5R)-2-(6-fluoro-1-benzothien-3-yl)bicyclo[3.2.1]oct-2-en-3-yl]methanamine hydrochloride,

N,N-dimethyl-[(1R,5S)-2-(6-fluoro-1-benzothien-3-yl)bicyclo[3.2.1]oct-2-en-3-yl]methanamine hydrochloride,

N,N-dimethyl-[(1S,5R)-2-(3-methyl-1-benzothien-7-yl)bicyclo[3.2.1]oct-2-en-3-yl]methanamine hydrochloride,

N,N-dimethyl-[(1S,5R)-2-(3-methyl-1-benzothien-7-yl)bicyclo[3.2.1]oct-2-en-3-yl]methanamine hydrochloride,

N,N-dimethyl-[(1S,5R)-2-(6-fluoro-naphth-2-yl)bicyclo [3.2.1]oct-2-en-3-yl]methanamine hydrochloride,

N,N-dimethyl-[(1R,5S)-2-(6-fluoro-naphth-2-yl)bicyclo [3.2.1]oct-2-en-3-yl]methanamine hydrochloride,

N-methyl-[3-(3-methyl-1-benzothien-7-yl)bicyclo [2.2.2]oct-2-en-2-yl]-methanamine hydrochloride,

N-methyl-[3-(6-fluoro-1-benzothien-3-yl)bicyclo [2.2.2]oct-2-en-2-yl]-methanamine hydrochloride,

N-methyl-[3-(1-benzothien-4-yl)bicyclo[2.2.2]oct-2-en-2-yl]-methanamine hydrochloride,

N-methyl-[3-(3-methyl-1-benzothien-5-yl)bicyclo [2.2.2]oct-2-en-2-yl]-methanamine hydrochloride,

N-methyl-[3-(1-benzothien-5-yl)bicyclo[2.2.2]oct-2-en-2-yl]-methanamine hydrochloride,

N-methyl-[2-(3-methyl-1-benzothien-5-yl)bicyclo [3.2.1]oct-2-en-3-yl]methanamine hydrochloride,

N-methyl-[2-(1-benzothien-7-yl)bicyclo[3.2.1]oct-2-en-3-yl]methanamine hydrochloride,

N-methyl-[2-(3-methyl-1-benzothien-7-yl)bicyclo [3.2.1]oct-2-en-3-yl]methanamine hydrochloride,

N-methyl-[2-(6-fluoro-1-benzothien-3-yl)bicyclo [3.2.1]oct-2-en-3-yl]methanamine hydrochloride,

N-methyl-[(1S,5R)-2-(6-fluoro-naphth-2-yl)bicyclo [3.2.1]oct-2-en-3-yl]methanamine hydrochloride,

N-methyl-[(1R,5S)-2-(6-fluoro-naphth-2-yl)bicyclo [3.2.1]oct-2-en-3-yl]methanamine hydrochloride,

N-methyl-[2-(1-benzothien-4-yl)bicyclo[3.2.1]oct-2-en-3-yl]methanamine hydrochloride,

N-methyl-[(1S,2R)-2-(6-cyano-1-benzothien-2-yl)bicyclo [3.2.1]oct-2-en-3-yl]methanamine hydrochloride,

N-methyl-[(1R,2S)-2-(6-cyano-1-benzothien-2-yl)bicyclo [3.2.1]oct-2-en-3-yl]methanamine hydrochloride,

N-methyl-[(1S,2R)-2-(1-benzothien-7-yl)bicyclo [3.2.1]oct-2-en-3-yl]methanamine hydrochloride,

N-methyl-[(1R,2S)-2-(1-benzothien-7-yl)bicyclo [3.2.1]oct-2-en-3-yl]methanamine hydrochloride,

N-methyl-[(1S,2R)-3-(3-methyl-1-benzothien-5-yl)bicyclo [2.2.2]oct-2-en-2-yl]-methanamine hydrochloride,

N-methyl-[(1R,2S)-3-(3-methyl-1-benzothien-5-yl)bicyclo [2.2.2]oct-2-en-2-yl]-methanamine hydrochloride,

N-methyl-[(1S,2R)-2-(1-benzothien-4-yl)bicyclo [3.2.1]oct-2-en-3-yl]methanamine hydrochloride,

N-methyl-[(1R,2S)-2-(1-benzothien-4-yl)bicyclo [3.2.1]oct-2-en-3-yl]methanamine hydrochloride,

N-methyl-2-(3-cyano-1-benzothien-5-yl)bicyclo[3.2.1]oct-2-en-3-yl]methanamine hydrochloride,

N-methyl-[2-(1-benzothien-2-yl)bicyclo[3.2.1]oct-2-en-3-yl]methanamine hydrochloride,

N-methyl-[2-(5-methyl-1-benzothien-2-yl)bicyclo [3.2.1]oct-2-en-3-yl]methanamine hydrochloride,

N-methyl-[2-(6-fluoro-1-benzothien-2-yl)bicyclo [3.2.1]oct-2-en-3-yl]methanamine hydrochloride,

N-methyl-[2-(7-fluoro-1-benzothien-4-yl)bicyclo [3.2.1]oct-2-en-3-yl]methanamine hydrochloride,

N-methyl-[2-(3-methyl-1-benzothien-4-yl)bicyclo [3.2.1]oct-2-en-3-yl]methanamine hydrochloride,

N-methyl-[2-(5-methylthio-1-benzothien-2-yl)bicyclo [3.2.1]oct-2-en-3-yl]methanamine hydrochloride,

N-methyl-[2-(1-benzofuran-7-yl)bicyclo[3.2.1]oct-2-en-3-yl]methanamine hydrochloride, and

N-methyl-[2-(1-benzofuran-6-yl)bicyclo[3.2.1]oct-2-en-3-yl]methanamine hydrochloride.

The invention also includes processes for the production of compounds of the formula (I) above. One process for producing the compounds of formula (I) comprises dehydrating a compound of formula

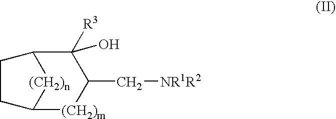

preferably employing a dehydrating agent, such as for example trifluoroacetic acid or p-toluene sulfonic acid. The reaction is preferably carried out in an organic solvent, such as for example dichloromethane, at a temperature of from 0° C. to 100° C.

Compounds of formula (II) can be prepared, for example, by reaction of a Grignard reagent of formula R³ MgBr, conveniently prepared in situ, with a cyclic ketone of the formula

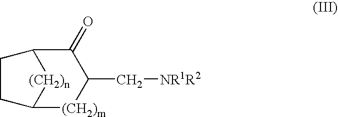

Alternatively, compounds of formula (II) can be prepared by reaction of a compound of formula (III) with a suitable lithium derivative of formula R³Li.

Such cyclic ketones can, in their turn, be prepared by reacting the appropriate amine of formula HNR¹R² with a compound of formula

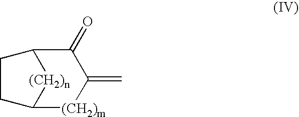

Compound (IV) can be made from the cyclic ketone by the Mannich reaction, to yield a compound of formula (III) in which R¹ and R² are both methyl, followed by quaternisation and elimination.

A further process for preparing compounds of formula (I), comprises reacting a compound of the formula

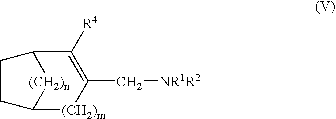

where R⁴ is a leaving group, with a suitable aryl metal complex that displaces R⁴ with the desired R³ group. The reaction is preferably carried out in an organic solvent at a temperature of from 0° C. to 100° C. A suitable leaving group is triflate.

It will be appreciated that substituents on the naphthyl or heterocyclyl ring can be introduced at the outset, or in the final stages of the synthesis. Sometimes it will be convenient to convert one substituent to another as, for example, C₁₋₄ alkoxy to hydroxy, at an intermediate stage or in the final product.

As mentioned above, the compounds of the invention and their pharmaceutically acceptable salts have useful central nervous system activity. The compounds inhibit the uptake of neurotransmitters such as serotonin, dopamine and noradrenalin. They are surprisingly effective serotonin reuptake inhibitors, as evidenced by their displacement of [³H] citalopram at the binding sites on membranes derived from rat cortex, as in the test described below (see Example 29). In a similar test, also employing rat cortex membrane, the compounds displaced nisoxetine, demonstrating their ability to inhibit noradrenalin reuptake, see Journal of Pharmacology and Experimental Therapeutics Vol. 272, No. 3, 1176-1186, 1995. The dopamine reuptake properties of the compounds of the invention are demonstrated in the test described in Molecular Pharmacology 45: 125-135, using membranes derived from rat striatum. In this test displacement of WIN 35,428 from its reuptake site, is measured.

Because of their profile of neurotransmitter reuptake properties, the compounds of the present invention are indicated for use in treating a variety of conditions such as depression, obesity, bulimia, alcoholism, pain, hypertension, ageing, senile dementia, Alzheimer's, memory loss, attention-deficit hyperactivity disorder, sexual dysfunction, Parkinsonism, anxiety, chronic fatigue syndrome, panic disorders, obsessive compulsive disorder, schizophrenia, gastrointestinal disorders, headache, cardiovascular disorders, smoking cessation, drug addiction including cocaine abuse, emesis and sleep disorders.

The compounds of the invention are effective over a wide dosage range, the actual dose administered being dependent on such factors as the particular compound being used, the condition being treated and the type and size of mammal being treated. However, the dosage required will normally fall within the range of 0.01 to 20 mg/kg per day, for example in the treatment of adult humans, dosages of from 0.5 to 100 mg per day may be used.

The compounds of the invention will normally be administered orally or by injection and, for this purpose, the compounds will usually be utilised in the form of a pharmaceutical composition. Such compositions are prepared in a manner well known in the pharmaceutical art and comprise at least one active compound.

Accordingly the invention includes a pharmaceutical composition comprising as active ingredient a compound of formula (I) or a pharmaceutically acceptable salt or ester thereof, associated with a pharmaceutically acceptable excipient. In making the compositions of the invention, the active ingredient will usually be mixed with a carrier, or diluted by a carrier, or enclosed within a carrier which may be in the form of a capsule, sachet, paper or other container. The excipient may be a solid, semi-solid or liquid material which acts as a vehicle, excipient or medium for the active ingredient. Some examples of suitable excipients are lactose, dextrose, sucrose, sorbitol, mannitol, starches, gum acacia, calcium phosphate, alginates, tragacanth, gelatin syrup, methyl cellulose, methyl- and propyl-hydroxybenzoate, talc, magnesium stearate or oil. The compositions of the invention may, if desired, be formulated so as to provide quick, sustained or delayed release of the active ingredient after administration to the patient.

Depending on the route of administration, the foregoing compositions may be formulated as tablets, capsules or suspensions for oral use and injection solutions or suspensions for parenteral use or as suppositories. Preferably the compositions are formulated in a dosage unit form, each dosage containing from 0.5 to 100 mg, more usually 1 to 100 mg, of the active ingredient.

The following Examples illustrate the synthesis of the compounds of invention.

### EXAMPLE 1

(±)-3-(N,N-Dimethylamino)methylbicyclo[2.2.1]heptan-2-one

To a solution of 81.8 mL of 2.0 M dimethylamine in THF (164 mmol) was added 3-methylene-bicyclo[2,2,1]heptan-2-one (10.0 mL, 81.9 mmol). The solution was stirred at room temperature for 24 h. The reaction mixture was then concentrated to give the crude title compound as an orange oil (13.50 g, 99% yield). IR (CHCl₃) 1732 cm⁻¹. Ion Spray MS 168 (M+H)⁺.

Similarly prepared were:

(1R/S,4R/S)-3-(N-pyrrolidyl)methylbicyclo [2.2.1]heptan-2-one, FDMS m/e=194 (M⁺+H).

(1R/S,4R/S)-3-(N-piperidinyl)methylbicyclo [2.2.1]heptan-2-one, FDMS m/e=208 (M⁺+H).

(1R/S,4R/S)-3-(N-morpholino) methylbicyclo [2.2.1]heptan-2-one, FDMS m/e=210 (M⁺+H).

(1R/S,4R/S)-3-(N-(4-tert-butyloxycarbonylpiperazinyl)) methylbicyclo[2.2.1]heptan-2-one, FDMS m/e=309 (M⁺+H).

(1R/S,4R/S)-3-(N-(4-methylpiperazinyl))methylbicyclo [2.2.1]heptan-2-one, FDMS m/e=223 (M⁺+H).

(1R/S,4R/S)-3-(N,N-diethyl) methylbicyclo [2.2.1]heptan-2-one, FDMS m/e=196 (M⁺+H).

CIS(1R/S,4R/S)-3-(N-(4-(2-(6-methoxy)naphthyl)-2-methyl piperidinyl))methylbicyclo[2.2.1]heptan-2-one, FDMS m/e=378 (M⁺+H)

trans-(1R/S,4R/S)-3-(N-(4-(2-(6-methoxy)naphthyl)-2-Methylpiperidinyl))methylbicyclo[2.2.1]heptan-2-one, FDMS m/e=378 (M⁺+H).

(1R/S,4R/S)-3-(N-(2-methyl)pyrrolidyl)methyl bicyclo[2.2.1]heptan-2-one, FDMS m/e=208 (M⁺+H).

(1R/S,4R/S)-3-(N-(2S-hydroxymethyl)pyrrolidyl) methylbicyclo[2.2.1]heptan-2-one, FDMS m/e=224 (M⁺+H).

(1R/S,4R/S)-3-(N-(2R-hydroxymethyl)pyrrolidyl) methylbicyclo[2.2.1]heptan-2-one, FDMS m/e=224 (M⁺+H).

(1R/S,4R/S)-3-(N-(2S-methoxymethyl)pyrrolidyl)methyl bicyclo[2.2.1]heptan-2-one, FDMS m/e=238 (M⁺+H).

(1R/S,4R/S)-3-(N-(2R-methoxymethyl) pyrrolidyl) methyl bicyclo[2.2.1]heptan-2-one, FDMS m/e=238 (M⁺+H).

(1R/S,4R/S)-3-(N-(3-hydroxy) pyrrolidyl) methyl bicyclo[2.2.1]heptan-2-one, FDMS m/e=210 (M⁺+H).

(1R/S,4R/S)-3-(N-(2,5-dimethyl)pyrrolidyl)methyl bicyclo[2.2.1]heptan-2-one, FDMS m/e=222 (M⁺+H).

(1R/S,4R/S)-3-(N-(3-S-acetamido)pyrrolidyl)methyl bicyclo[2.2.1]heptan-2-one, FDMS m/e=251 (M⁺+H).

### EXAMPLE 2

(+/−)-3-(N,N-Dimethylamino)methyl-2-(4-methoxybenzo[b]thiophen-2-yl)bicyclo[2.2.1]heptan-2-ol

To a solution of 4-methoxybenzo[b]thiophene (0.500 g, 3.04 mmol) in dry THF (10.0 mL) at −78° C. was added 1.6 M n-BuLi in hexanes (2.28 mL, 3.65 mmol). The solution was stirred at −78° C. for 45 min and then 3-(N,N-dimethylamino)methyl[2,2,1]heptan-2-one (0.509 g, 3.04 mmol) in THF (3 mL) was added via a cannulla at −78° C. The reaction mixture was stirred at −78° C. for 1.75 h. The reaction was then quenched with 15 mL of saturated aqueous NH₄Cl solution. The mixture was extracted with EtOAc (2×100 mL). The combined organic layers were dried over MgSO₄ and filtered. The filtrate was concentrated and purified by medium pressure chromatography (silica gel, 2% (2.0 M NH₃ in MeOH)/CH₂Cl₂) to give the title compound as a white solid (0.283 g, 28%). IR (KBr) 3100 (br) cm⁻¹. EI⁺ MS 228 (M−103)⁺(base peak); 331 (M)⁺.

Similarly prepared were:

(1R/S,4R/S)-3-(N-pyrrolidyl)methyl-2-(2-naphthyl)bicyclo [2.2.1]heptan-2-ol FDMS m/e=322 (M⁺+H).

(1R/S,4R/S)-3-(N-piperidinyl)methyl-2-(2-naphthyl) bicyclo[2.2.1]heptan-2-ol, FDMS m/e=336 (M⁺+H).

(1R/S,4R/S)-3-(N-morpholino)methyl-2-(2-naphthyl)bicyclo [2.2.1]heptan-2-ol, FDMS m/e=338 (M⁺+H).

(1R/S,4R/S)-3-(N-(4-tert-butyloxycarbonylpiperazinyl)) methyl-2-(2-naphthyl)bicyclo[2.2.1]heptan-2-ol, FDMS m/e=437 (M⁺+H).

(1R/S,4R/S)-3-(N-(4-methylpiperazinyl))methyl-2-(2-naphthyl)bicyclo[2.2.1]heptan-2-ol, FDMS m/e=351 (M⁺+H).

(1R/S,4R/S)-3-(N,N-diethylamino)methyl-2-(2-naphthyl) bicyclo[2.2.1]heptan-2-ol, FDMS m/e=324 (M⁺+H).

cis-(1R/S,4R/S)-3-(N-(4-(2-(6-methoxy)naphthyl)-2-methylpiperidinyl))methyl-2-(2-naphthyl)bicyclo[2.2.1]heptan-2-ol, FDMS m/e=506 (M⁺+H).

trans-(1R/S,4R/S)-3-(N-(4-(2-(6-methoxy)naphthyl)-2-methylpiperidinyl))methyl-2-(2-naphthyl)bicyclo (2.2.1]heptan-2-ol, FDMS m/e=506 (M⁺+H).

(1R/S,4R/S)-3-(N-(2-methyl)pyrrolidyl)methyl-2-(2-naphthyl)bicyclo[2.2.1]heptan-2-ol, FDMS m/e=336 (M⁺+H).

3-(N-(2S-hydroxymethyl)pyrrolidyl) methyl-2-(2-naphthyl) bicyclo[2.2.1]heptan-2-ol, FDMS m/e=352 (M⁺+H).

3-(N-(2R-hydroxyymethyl)pyrrolidyl) methyl-2-(2-naphthyl) bicyclo[2.2.1]heptan-2-ol, FDMS m/e=352 (M⁺+H).

(1R/S,4R/S)-3-(N-(2S-methoxymethyl)pyrrolidyl) methyl-2-(2-naphthyl)bicyclo[2.2.1]heptan-2-ol, FDMS m/e=366 (M⁺+H).

(1R/S,4R/S)-3-(N-(2R-methoxymethyl)pyrrolidyl) methyl-2-(2-naphthyl)bicyclo[2.2.1]heptan-2-ol, FDMS m/e=366 (M⁺+H).

(1R/S,4R/S)-3-(N-(3-hydroxy)pyrrolidyl) methyl-2-(2-naphthyl)bicyclo[2.2.1]heptan-2-ol, MS m/e=338 (M⁺+H).

(1R/S,4R/S)-3-(N-(2,5-dimethyl)pyrrolidyl)methyl-2-(2-naphthyl)bicyclo[2.2.1]heptan-2-ol, FDMS m/e=350 (M⁺+H).

3-(N-(3-S-acetamido)pyrrolidyl)methyl-2-(2-naphthyl) bicyclo[2.2.1]heptan-2-ol, FDMS m/e=379 (M⁺+H).

### EXAMPLE 3

(+/−) Trifluoromethanesulfonic Acid 3-Dimethylaminomethylbicyclo[2.2.1]hept-2-en-2-yl Ester.

To a cooled (−78° C.) solution of diisopropylamine (11.8 mL, 84.5 mmol) in anhydrous THF (85 mL) was added 1.6 M n-BuLi in hexanes (52.8 mL, 84.5 mmol). The solution was stirred at −78° C. for 1 h. The dry ice bath was removed and the solution was stirred for an additional 0.5 h. The solution was then recooled to −78° C. and (+−)-3-(N,N-dimethylamino)methylbicyclo [2.2.1]heptan-2-one (13.5 g, 80.5 mmol) in anhydrous THF (85 mL) was added via a cannulla over a period of 15 min. The solution was stirred at −78° C. for 35 min. N-Phenyl-bis(trifluoromethanesulfonimide) (30.2 g, 84.5 mmol) in anhydrous THF (85 mL) was added to the reaction mixture via a canulla over a period of 20 min. at −78° C. The resulting solution was allowed to slowly warm to room temperature overnight. The reaction mixture was then concentrated under reduced pressure. The residue was diluted with CH₂Cl₂ (75 mL). The mixture was then eluted on a pad of neutral alumina using 5% EtOAc in hexanes (3 L) as eluent. The collected fraction was concentrated and purified by medium pressure chromatography (silica gel, 1.5-2.0% (3.5 M NH₃ in MeOH)/CH₂Cl₂) to give the title compound as a yellow oil (17.4 g, 72%). IR (CHCl₃) 1419, 1141 cm⁻¹. Ion Spray MS 300 (M+H)⁺.

### EXAMPLE 4

(+/−)(N,N-Dimethylamino)methyl-2-(6-fluorobenzo[b]thiophen-3-yl)bicyclo[2.2.1]heptan-2-ol

To a solution of crude 3-bromo-6-fluorobenzo[b]thiophene (0.960 g, 4.15 mmol) in anhydrous THF (20 mL) was added dibromoethane (0.358 ml, 4.15 mmol) and magnesium turnings (0.202 g, 8.30 mmol). The solution was stirred at reflux for 2 h. or until the magnesium was consumed. The reaction mixture was removed from the heat and (+−)(N,N-dimethylamino)methylbicyclo[2.2.1]heptan-2-one (0.764 g, 4.57 mmol) in anhydrous THF (6 mL) was added to the solution at room temperature. The solution was stirred for 20 h. The reaction was quenched with saturated aqueous NH₄Cl solution. (30 mL). The mixture was extracted with EtOAc (2×100 mL). The combined organic layers were dried over MgSO₄ and concentrated. The residue was purified by medium pressure chromatography (silca gel, 2.5% (2.0 M NH₃ in MeOH)/CH₂Cl₂) to give the title compound as a white semisolid (0.271 g, 20%). Ion Spray MS 320 (M+H)⁺, 290 (base peak) (M−29)⁺.

### EXAMPLE 5

3-[(Dimethylamino)methyl]-2-(6-fluoro-2-naphthyl)bicyclo [2.2.1]heptan-2-ol

The enantiomers of 3-[(dimethylamino) methyl]-2-(6-fluoro-2-naphthyl)bicyclo[2.2.1]heptan-2-ol were separated by chiral preparative chromatography, using a CHIRALPAK-AD column, and a mixture of Hexane/Ethanol 80/20 as the eluant.

### EXAMPLE 6

(+/−)Dimethyl-[3-(4-methoxybenzo[b]thiophen-2-yl)bicyclo[2.2.1]hept-2-en-2-yl]methylamine hydrochloride

To a solution of (+/−) (N,N-dimethylamino)methyl-2-(4-methoxybenzo[b]thiophen-2-yl)bicyclo [2.2.1]heptan-2-ol (0.277 g, 0.835 mmol) in dry CH₂Cl₂ (7 mL) at 0° C. was added 2 mL of trifluoroacetic acid. The solution was stirred at 0° C. for 3 h. The reaction was then quenched with saturated aqueous NaHCO₃ solution (30 mL). The mixture was extracted (2×40 mL) with CH₂Cl₂. The combined organic layers were dried over MgSO₄ and concentrated. The residue was purified by medium pressure chromatography (silica gel, 3% (2.0 M NH₃ in MeOH)/CH₂Cl₂) to give the free base of the title compound as an off-white semi-solid (0.243 g, 93%). The hydrochloride salt was prepared with 1.0 equiv. of HCl in EtOAc. m.p. (HCl) 239-242° C. FDMS m/e=313 M⁺.

Similarly prepared were:

N,N-dimethyl[3-(2-naphthyl)bicyclo[2.2.1]hept-2-en-2-yl]methanamine hydrochloride, mp 226.3-227.3° C.

[3-(1-benzothien-2-yl)bicyclo[2.2.1]hept-2-en-2-yl]-N,N-dimethylmethanamine hydrochloride, FIA POS MS (M+H, 284)

[3-(3-bromo-1-benzothien-2-yl)bicyclo[2.2.1]hept-2-en-2-yl]-N,N-dimethylmethanamine hydrochloride, mp 1090, FIA POS MS (M+H, 362/364)

[3-(6-methoxy-2-naphthyl)bicyclo[2.2.1]hept-2-en-2-yl]-N,N-dimethylmethanamine hydrochloride, mp 201.7-202.5° C.

6-(3-[(dimethylamino)methyl]bicyclo[2.2.1]hept-2-en-2-yl}-2-naphthol hydrochloride, mp 222.9-224.5° C.

[3-(6-bromo-2-naphthyl)bicyclo[2.2.1]hept-2-en-2-yl]-N,N-dimethylmethanamine hydrochloride, mp 225.8-226.6° C.

[3-(1-benzofuran-2-yl)bicyclo[2.2.1]hept-2-en-2-yl]-N,N-dimethylmethanamine hydrochloride, mp 241.5-242.6° C.

[3-(6-fluoro-2-naphthyl)bicyclo[2.2.1]hept-2-en-2-yl]-N,N-dimethylmethanamine hydrochloride, mp 223.4-224.5° C.

(1-benzothien-5-yl)bicyclo[2.2.1]hept-2-en-2-yl]-N,N-dimethylmethanamine hydrochloride, mp 227.5-228.5° C.

[3-(1-benzothien-3-yl)bicyclo[2.2.1]hept-2-en-2-yl]-N,N-dimethylmethanamine hydrochloride, mp 205.0-206.6° C.

N,N-dimethyl[3-(3-quinolinyl)bicyclo[2.2.1]hept-2-en-2-yl]methanamine dihydrochloride, mp 246.4-248.9° C.

N,N-dimethyl[3-(5-methyl-1-benzothien-2-yl)bicyclo[2.2.1]hept-2-en-2-yl]methanamine hydrochloride, mp 231.6-232.5° C.

N,N-dimethyl[3-(6-methyl-2-naphthyl)bicyclo[2.2.1]hept-2-en-2-yl]methanamine, maleic acid, mp 171-172° C.

[3-(1-benzothien-6-yl)bicyclo[2.2.1]hept-2-en-2-yl]-N,N-dimethylmethanamine,, maleic acid, mp 185-187° C.

[3-(6-chloro-1,3-benzothiazol-2-yl)bicyclo[2.2.1]hept-2-en-2-yl]-N,N-dimethylmethanamine succinate, FIA POS MS (M+H, 321.0)

N,N-dimethyl{3-[6-(methylsulfanyl)-2-naphthyl]bicyclo[2.2.1]hept-2-en-2-yl}methanamine, maleic acid, MS ESP+ (M+,324.1)

[3-(6-methoxy-1-benzothien-2-yl)bicyclo[2.2.1]hept-2-en-2-yl]-N,N-dimethylmethanamine hydrochloride, Ion Spray MS 269 (M-44(-N(CH₃)₂))⁺

[3-(5-methoxy-1-benzothien-2-yl) bicyclo [2.2.1]hept-2-en-2-yl]-N,N-dimethylmethanamine hydrochloride, FDMS m/e=314 M⁺

[3-(8-methoxy-2-naphthyl)bicyclo[2.2.1]hept-2-en-2-yl]-N,N-dimethylmethanamine, maleic acid, MS ESP+ (M+,308.1)

N,N-dimethyl [3-(2-quinolinyl)bicyclo[2.2.1]hept-2-en-2-yl]methanamine succinate, MS ESP+ (M+279.0)

[3-(4-isoquinolinyl)bicyclo[2.2.1]hept-2-en-2-yl]-N,N-dimethylmethanamine succinate, MS ESP+ (M+279.0)

N,N-dimethyl[3-(6-quinolinyl)bicyclo[2.2.1]hept-2-en-2-yl]methanamine dihydrochloride, MS ESP+ (M+279.0)

N,N-dimethyl[3-(2-quinolinyl)bicyclo[2.2.1]hept-2-en-2-yl]methanamine dihydrochloride, MS ESP+ (M+279.0)

[3-(4-isoquinolinyl)bicyclo[2.2.1]hept-2-en-2-yl]-N,N-dimethylmethanamine dihydrochloride, MS ESP+ (M+279.0)

N,N-dimethyl[3-(8-quinolinyl)bicyclo[2.2.1]hept-2-en-2-yl]methanamine dihydrochloride, MS ESP+ (M+279.0)

N,N-dimethyl[3-(5-quinolinyl)bicyclo[2.2.1]hept-2-en-2-yl]methanamine dihydrochloride, MS ESP+ (M+279.0)

[3-(6-fluoro-1-benzothien-3-yl)bicyclo[2.2.1]hept-2-en-2-yl]-N,N-dimethylmethanamine hydrochloride, Ion Spray MS 302 (M+H)⁺, 257 (M-44(—N(CH₃)₂))⁺

N,N-dimethyl[3-(2-methyl-6-quinolinyl)bicyclo[2.2.1]hept-2-en-2-yl]methanamine dihydrochloride, MS ESP+ (M+, 293.0)

N,N-dimethyl[3-(4-methyl-2-quinolinyl)bicyclo[2.2.1]hept-2-en-2-yl]methanamine dihydrochloride, MS ESP+ (M+,293.2)

N,N-dimethyl [3-(2-methyl-1,3-benzothiazol-5-yl)bicyclo[2.2.1]hept-2-en-2-yl]methanamine hydrochloride, FIA POS MS (M+H,299.2)

[3-(5-methoxy-2-naphthyl)bicyclo[2.2.1]hept-2-en-2-yl]-N,N-dimethylmethanamine, maleic acid, MS ESP+ (M+,308.2)

[3-(6-fluoro-1,3-benzothiazol-2-yl)bicyclo[2.2.1]hept-2-en-2-yl]-N,N-dimethylmethanamine hydrochloride, MS (M+H,303.1)

[3-(4-chloro-1,3-benzothiazol-2-yl)bicyclo[2.2.1]hept-2-en-2-yl]-N,N-dimethylmethanamine hydrochloride, MS ESP+ (M+,319.0)

[3-(6-methoxy-1,3-benzothiazol-2-yl)bicyclo[2.2.1]hept-2-en-2-yl]-N,N-dimethylmethanamine hydrochloride, FIA POS MS (M+H,315.3)

N,N-dimethyl[3-(1-naphthyl)bicyclo[2.2.1]hept-2-en-2-yl]methanamine, maleic acid, mp 142.9-143.7° C.

[3-(6-chloro-1-benzothien-2-yl)bicyclo[2.2.1]hept-2-en-2-yl]-N,N-dimethylmethanamine hydrochloride, mp 235-237° C.

[3-(4-methoxy-1,3-benzothiazol-2-yl)bicyclo[2.2.1]hept-2-en-2-yl]-N,N-dimethylmethanamine hydrochloride, MS ESP+ (M+,314.0)

[3-(7-bromo-4-methoxy-1,3-benzothiazol-2-yl)bicyclo[2.2.1]hept-2-en-2-yl]-N,N-dimethylmethanamine hydrochloride, FIA POS MS (M+H,395.0)

N,N-dimethyl{3-[6-(trifluoromethyl)-1-benzothien-2-yl]bicyclo[2.2.1]hept-2-en-2-yl}methanamine hydrochloride, mp 237-239° C.

[3-(6-chloro-1-benzothien-3-yl)bicyclo[2.2.1]hept-2-en-2-yl]-N,N-dimethylmethanamine hydrochloride, FIA Pos MS (M+H, 318.1)

N,N-dimethyl{3-[6-(trifluoromethyl)-1-benzothien-3-yl]bicyclo[2.2.1]hept-2-en-2-yl}methanamine hydrochloride, mp 169-171° C.

[3-(4-fluoro-1-naphthyl)bicyclo[2.2.1]hept-2-en-2-yl]-N,N-dimethylmethanamine hydrochloride, mp 245.5-247.4° C.

N,N-dimethyl[3-(6-methyl-1-benzothien-2-yl)bicyclo[2.2.1]hept-2-en-2-yl]methanamine hydrochloride, mp 228-230° C.

1-{[3-(2-naphthyl)bicyclo[2.2.1]hept-2-en-2-yl]methyl}piperidine hydrochloride, FDMS m/e=318 (M⁺+H of free base).

4-{[(1R,4S)-3-(2-naphthyl)bicyclo[2.2.1]hept-2-en-2-yl]methyl}morpholine hydrochloride, FDMS m/e=320 (M⁺+H of free base).

1-methyl-4-{[3-(2-naphthyl)bicyclo[2.2.1]hept-2-en-2-yl]methyl}piperazine hydrochloride, FDMS m/e=333 (M⁺+H of free base).

1-{[3-(2-naphthyl)bicyclo[2.2.1]hept-2-en-2-yl]methyl}piperazine dihydrochloride, FDMS m/e=319 (M⁺+H of free base).

N-ethyl-N-{[3-(2-naphthyl)bicyclo[2.2.1]hept-2-en-2-yl]methyl}ethanamine hydrochloride, FDMS m/e=305(M⁺ of free base).

4-(6-methoxy-2-naphthyl)-2-methyl-1-{[(1R,4S)-3-(2-naphthyl)bicyclo[2.2.1]hept-2-en-2-yl]methyl}piperidine hydrochloride, FDMS m/e=488(M⁺ of free base).

4-(6-methoxy-2-naphthyl)-2-methyl-1-{[(1R,4S)-3-(2-naphthyl)bicyclo[2.2.1]hept-2-en-2-yl]methyl)piperidine hydrochloride, FDMS m/e=488(M⁺ of free base).

2-methyl-1-{[3-(2-naphthyl)bicyclo[2.2.1]hept-2-en-2-yl]methyl}pyrrolidine hydrochloride, FDMS m/e=318 (M⁺+H of free base).

Methyl ((2R)-1-{[3-(2-naphthyl)bicyclo[2.2.1]hept-2-en-2-yl]methyl}-2-pyrrolidinyl)methyl ether hydrochloride, FDMS m/e=348 (M⁺+H of free base). α[D]₅₈₉=160 (c=0.5, methanol).

Methyl ((2S)-1-{[3-(2-naphthyl)bicyclo[2.2.1]hept-2-en-2-yl]methyl}-2-pyrrolidinyl)methyl ether hydrochloride, FDMS m/e=348 (M⁺+H of free base). α[D]₅₈₉=−171.4(c=0.56, methanol).

((2S)-1-{[3-(2-naphthyl)bicyclo[2.2.1]hept-2-en-2-yl]methyl}-2-pyrrolidinyl)methanol hydrochloride, FDMS m/e=334 (M⁺+H of free base). α[D]₅₈₉=−183(c=0.59 methanol).

((2R)-1-{[3-(2-naphthyl)bicyclo[2.2.1]hept-2-en-2-yl]methyl}-2-pyrrolidinyl)methanol hydrochloride, FDMS m/e=334 (M⁺+H of free base). α[D]₅₈₉=−15.09(c=0.53, methanol).

N-(2,5-dimethyl)pyrrolidyl-[(1R/S,4R/S)-2-(2-naphthyl)bicyclo[2.2.1]-2-hepten-3-yl]methylamine hydrochloride, FDMS m/e=332 (M⁺+H of free base).

N-(2R-hydroxymethyl)pyrrolidyl-[2-(2-naphthyl)bicyclo [2.2.1]-2-hepten-3-yl]methylamine hydrochloride, FDMS m/e=334 (M⁺+1 of free base). α[D]₅₈₉=182.8 (c=0.58 methanol).

N-(2R-hydroxymethyl)pyrrolidyl-[2-(2-naphthyl)bicyclo [2.2.1]-2-hepten-3-yl]methylamine hydrochloride, FDMS m/e=334 (M⁺+H of free base). α[D]₅₈₉=17.86 (c=0.56 methanol).

1-{[3-(2-naphthyl)bicyclo[2.2.1]hept-2-en-2-yl]methyl}-3-pyrrolidinol hydrochloride, FDMS m/e=320 (M⁺+H of free base).

N-((3S)-1-{[3-(2-naphthyl)bicyclo[2.2.1]hept-2-en-2-yl]methyl}-3-pyrrolidinyl)acetamide hydrochloride, FDMS m/e=361 (M⁺+H of free base). α[D]₅₈₉=−49.06 (c=0.53, methanol). Mp 122-124° C.

N-((3R)-1-{[3-(2-naphthyl)bicyclo[2.2.1]hept-2-en-2-yl]methyl}-3-pyrrolidinyl)acetamide hydrochloride, FDMS m/e=361 (M⁺+H of free base). α[D]₅₈₉=50.10 (c=0.50, methanol). Mp 124-126° C.

1-{[3-(6-fluoro-1-benzothien-3-yl)bicyclo[2.2.1]hept-2-en-2-yl]methyl}pyrrolidine hydrochloride, FIA POS MS (M+H, 328.2)

1-{[3-(3-methyl-1-benzothien-5-yl)bicyclo[2.2.1]hept-2-en-2-yl]methyl}pyrrolidine hydrochloride, FIA POS MS (M+H, 324.1),

2,2-dimethyl-1-{[3-(2-naphthyl)bicyclo[2.2.1]hept-2-en-2-yl]methyl}pyrrolidine hydrochloride, FIA POS MS (M+H, 332.2),

N-{[3-(1-benzothien-7-yl)bicyclo[2.2.1]hept-2-en-2-yl]methyl}-N-methyl-2-propen-1-amine hydrochloride, FIA POS MS (M+H 310.1),

[3-(1-benzothien-7-yl)bicyclo [2.2.1]hept-2-en-2-yl]-N-methylmethanamine hydrochloride, FIA POS MS (M+H, 270.1)

N,N-pyrrolidyl-[(1R/S,4R/S)-2-(2-naphthyl)bicyclo [2.2.1]-2-hepten-3-yl]methylamine hydrochloride, FDMS m/e=304 (M⁺+H of free base)

### EXAMPLE 7

(+/−)-Dimethyl-[3-(5-fluorobenzo[b]thiophen-2-yl) bicyclo [2.2.1]hept-2-en-2-yl]methylamine hydrochloride.

To a cooled solution (−78° C.) of 5-fluorobenzo [b]thiophene (1.00 g, 6.57 mmol) in anhydrous THF (20 mL) was added 1.6 M n-BuLi in hexanes (4.11 mL, 6.57 mmol). The resulting solution was stirred at −78° C. for 45 min. The cold bath was removed and 0.5 M zinc chloride in toluene (13.1 mL, 6.57 mmol) was added and the mixture was stirred for 15 min. A slurry of (+−)-trifluoromethanesulfonic acid 3-dimethylaminomethyl bicyclo[2.2.1]hept-2-en-2-yl ester (1.48 g, 4.93 mmol), triphenylarsine (0.201 g, 0.657 mmol) and tris (dibenzylideneacetone)dipalladium(0) (0.301 g, 0.329 mmol) in anhydrous DMF (20 mL) was added to the reaction mixture via a canulla at room temperature. The reaction mixture was heated to reflux and stirred for 3 days. The reaction mixture was then cooled to room temperature and concentrated under reduced pressure. Xylenes were used to remove azeotropically the residual DMF. The residue was then partitioned between CH₂Cl₂ (70 mL) and H₂O (20 mL). The layers were separated, and the aqueous layer was extracted with CH₂Cl₂ (1×100 mL) The combined organic layers were dried over MgSO₄ and concentrated. The residue was purified by medium pressure chromatography (silica gel, 2% (2.0 M NH₃ in MeOH)/CH₂Cl₂) to give the free base of the title compound as a clear oil (0.416 g, 28%). The hydrochloride salt was prepared with 1.0 equiv. of HCl in EtOAc. Ion Spray MS (HCl) 302 (M+H)⁺, 257 (M-44(—N(CH₃)₂))⁺.

Similarly prepared were:

(+/−)-dimethyl-[3-(4-fluorobenzo[b]thiophen-2-yl)bicyclo[2.2.1]hept-2-en-2-yl]methylamine Hydrochloride, Ion Spray MS (HCl) 302 (M+H)⁺, 257 (M-44(—N(CH₃)₂))⁺

(+/−)-dimethyl-[3-(6-fluorobenzo[b]thiophen-2-yl)bicyclo[2.2.1]hept-2-en-2-yl]methylamine Hydrochloride, FDMS m/e=301 M⁺

(+/−)-dimethyl-[3-(7-fluorobenzo[b]thiophen-2-yl)bicyclo[2.2.1]hept-2-en-2-yl]methylamine Hydrochloride, Ion Spray MS (HCl) 302 (M+H)⁺, 257 (base peak) (M-44(—N(CH₃)₂))⁺, 258 (M-43)⁻

### EXAMPLE 8

(+/−)-Dimethyl-[3-(7-methoxybenzo[b]thiophen-2-yl)bicyclo[2.2.1]hept-2-en-2-yl]methylamine hydrochloride

A slurry containing 7-methoxybenzo[b]thiophene-2-boronic acid (0.500 g, 2.40 mmol), trifluoromethane sulfonic acid 3-dimethylaminomethylbicyclo[2.2.1]hept-2-en-2-yl ester (0.504 g, 1.68 mmol), lithium chloride (0.214 g, 5.05 mmol), tetrakis(triphenylphosphine) palladium(0) (0.099, 0.097 mmol) and 2.0 N Na₂CO₃ (2.40 mL, 4.81 mmol) in 1,2-dimethoxyethane (6 mL) was heated to reflux and stirred for 2 h. The reaction mixture was then cooled to room temperature and concentrated under reduced pressure. The residue was dissolved in CH₂Cl₂ (20 mL), 2.0 N Na₂CO₃ (20 mL) and conc. NH₄OH (1 mL) The layers were separated and the aqueous layer was extracted with CH₂Cl₂ (1×30 mL). The organic layers were combined and dried over MgSO₄ and concentrated. The residue was purified by medium pressure chromatography (silica gel, 1-3% (3.5 M NH₃ in MeOH)/CH₂Cl₂) to give the free base of the title compound as a brown semi-solid (0.036 g, 7%). The hydrochloride salt was prepared with 1.0 equiv. of HCl in EtOAc. m.p. 232-235° C.; FDMS m/e=313 M⁺.

### EXAMPLE 9

(+/−)-3-(N-Benzyl-N-methylamino)methylbicyclo[2.2.1]heptan-2-one

To a solution of 3-methylene-bicyclo[2.2.1]heptan-2-one (6.00 mL, 49.0 mmol) in anhydrous THF (55 mL) was added N-benzylmethylamine. The solution was stirred at room temperature for 7 days. The reaction mixture was then concentrated under reduced pressure. The residue was purified by medium pressure chromatography (silica gel, 2% (2.0 M NH₃ in MeOH)/CH₂Cl₂) to give the title compound as a yellow oil (11.48 g, 96%). Ion Spray MS 244 (M+H)⁺

### EXAMPLE 10

(+/−)-3-(N-Benzyl-N-methylamino)methyl-2-(6-fluorobenzo[b]thiophen-2-yl)bicyclo[2.2.1]heptan-2-ol.

To a solution of a 1:6 mixture of 4- and 6-fluorobenzo[b]thiophene (1.00 g, 6.57 mmol) and TMEDA (4.96 mL, 32.9 mmol) in dry THF (20 mL) at −78° C. was added 1.6 M n-BuLi in hexanes (4.52 mL, 7.23 mmol). The solution was stirred at −78° C. for 55 min, and then 3-(N-benzyl-N-methylamino)methylbicyclo[2.2.1]heptan-2-one (1.60 g, 6.57 mmol) in THF (4 mL) was added via a canulla at −78° C. The resulting bright red reaction mixture was stirred at −78° C. for 1.75 h. The reaction was then quenched with 0.30 mL of saturated aqueous NH₄Cl solution. The mixture was extracted with CH₂Cl₂ (2×100 mL). The combined organic layers were dried over MgSO₄ and filtered. The filtrate was concentrated and purified by medium pressure chromatography (silica gel, 5% EtOAc in hexanes) to give the title compound as a clear semi-solid (1.27 g, 49%). IR (CHCl₃) 3200 (br) cm¹ Ion Spray MS 396 (M+H)⁺.

### EXAMPLE 11

(+/−)-2-(6-Fluorobenzo[b]thiophen-2-yl)-3-(N-methylamino)methylbicyclo[2.2.1]heptan-2-ol

To a solution of (+−)-3-(N-benzyl-N-methylamino)methyl-2-(6-fluorobenzo[b]thiophen-2-yl)bicyclo[2.2.1]heptan-2-ol (292 mg, 0.738 mmol) in a 1:1 THF:EtOH mixture (5 mL) was added 10% Pd/C (300 mg). The black slurry was stirred vigorously under an H₂ atmosphere for 4 days. The slurry was diluted with EtOH, filtered over a pad of diatomaceous earth, and washed with EtOH. The filtrate was concentrated and the residue was purified by medium pressure chromatography (silica gel, 3% (2.0 M NH₃ in MeOH)/CH₂Cl₂) to give the title compound as a white solid (0.031 g, 14%). Ion Spray MS 306 (M+H)⁺, 245 (base peak) (M-60)⁺.

### EXAMPLE 12

(+/−)-((3-(6-Fluorobenzo[b]thiophen-2-yl)bicyclo[2.2.1]hept-2-en-2-yl)methyl)methylamine hydrochloride

To a solution of (+−)-2-(6-fluorobenzo[b]thiophen-2-yl)-3-(N-methylamino)methylbicyclo[2.2.1]heptan-2-ol (0.029 g, 0.093 mmol) in dry CH₂Cl₂ (1.0 mL) at 0° C. was added 0.4 mL of trifluoroacetic acid. The solution was stirred at 0° C. for 2 h. The reaction was then quenched with saturated aqueous NaHCO₃ solution (7 mL). The mixture was extracted with CH₂Cl₂ (2×25 mL). The combined organic layers were dried over MgSO₄ and concentrated. The residue was purified by medium pressure chromatography (silica gel, 3% (2.0 M NH₃ in MeOH)/CH₂Cl₂) to give the free base of the title compound as a brown semi-solid (0.021 g, 78%). The hydrochloride salt was prepared with 1.0 equiv. of HCl in EtOAc. FDMS (HCl) m/e=287 M⁺

Similarly prepared were:

[(1R,4S)-3-(1,3-benzothiazol-2-yl)bicyclo [2.2.1]hept-2-en-2-yl]-N-methylmethanamine succinate, MS ESP+ (M+, 305.0)

[(1R,4S)-3-(6-chloro-1,3-benzothiazol-2-yl)bicyclo[2.2.1]hept-2-en-2-yl]-N-methylmethanamine succinate, FIA POS MS (M+H 271.0)

N-benzyl-N-{[3-(6-methoxy-2-naphthyl)bicyclo[2.2.1]hept-2-en-2-yl]methyl}-N-methylamine hydrochloride, mp 178-180° C.

### EXAMPLE 13

(4-Bromobenzo[b]thiophen-2-yl)trimethylsilane

Into a stirred solution of diisopropylamine (1.08 mL, 7.74 mmol) and TMEDA (2.54 mL, 16.89 mmol) in THF (15 mL, freshly distilled) at 0° C. was added 1.6 M n-BuLi (5.27 mL, 8.44 mmol) via a syringe. The solution was stirred for 30 min at 0° C. and was then cooled to −78° C., and a solution of 4-bromobenzo[b]thiophene (1.5 g, 7.03 mmol) in THF (7 mL) was added via a cannula. After stirring for 30 min, a solution of TMSCl (1.07 mL, 8.44 mmol) in THF (8 mL) was added via a cannula. The solution was stirred overnight, while allowed to warm to room temperature. The solution was then concentrated under reduced pressure. Saturated aqueous NaHCO₃ (100 mL) was added and the mixture was extracted with CH₂Cl₂ (4×80 mL). The organic layers were combined, dried over MgSO₄, filtered and concentrated under reduced pressure. The residue was purified by silica gel chromatography [100% hexanes]. The purified fractions were combined and concentrated under reduced pressure to give (4-bromobenzo[b]thiophen-2-yl)trimethylsilane as a clear oil (1.7458 g, 87%). IR (KBr) 2959, 972, 853, 838 cm⁻¹. EI MS 286.2 (M+H)⁺, 271.1 (M-CH₃)⁺

### EXAMPLE 14

(+/−)-3-(N,N-Dimethylamino)methyl-2-(2-trimethyl silylbenzo[b]thiophen-4-yl)bicyclo[2.2.1]heptan-2-ol

Into a stirred solution of diisopropylamine (0.493 mL, 3.52 mmol) and TMEDA (1.16 mL, 7.689 mmol) in THF (6.5 mL, freshly distilled) at −78° C. was added 1.6 M n-BuLi (2.40 mL, 3.84 mmol) via a syringe. The solution was warmed to 0° C. for 20 min and re-cooled to −78° C. A solution of 4-bromobenzo[b]thiophene (0.6828 g, 3.204 mmol) in THF (3.25 mL) was added via a cannula. After stirring for 20 min, a solution of TMSCl (0.488 mL, 3.84 mmol) in THF (3.25 mL) was added via a cannula. The solution was stirred overnight, while allowed to warm to room temperature. Saturated aqueous NH₄Cl (10 mL) was added and the mixture was extracted with Et₂O (3×15 mL). The organic layers were combined, dried over MgSO₄, filtered and concentrated under reduced pressure to give 4-bromo-2-trimethylsilylbenzo[b]thiophene as a gold oil (0.845 g, 92%).

Into a solution of crude 4-bromo-2-trimethylsilylbenzo[b]thiophene (0.500 g, 1.75 mmol) and TMEDA (1.32 mL, 8.76 mmol) in THF (5 mL) cooled at −78° C. was added 1.6 M n-BuLi (1.20 mL, 1.927 mmol) via a syringe. After stirring for 30 min, a solution of (+/−)-3-(N,N-dimethylamino)methylbicyclo[2.2.1]heptan-2-one, (0.293 g, 1.75 mmol) in THF (2 mL) was added via a cannula. After stirring overnight, the solution was diluted with H₂O (25 mL) and saturated aqueous NH₄Cl (25 mL). The mixture was extracted with EtOAc (3×50 mL), dried over MgSO₄, filtered and concentrated under reduced pressure. The residue was then purified by silica gel chromatography (2% (2.0 M NH₃ in MeOH)/CH₂Cl₂). The purified fractions were concentrated under reduced pressure and placed under vacuum to give the title compound as a yellow oil. (0.3351 g, 51%). IR (KBr) 3005, 2959, 1251, 993, 842 cm⁻¹. Ion Spray MS 374.2 (M+H)⁺.

### EXAMPLE 15

(+/−)-((3-(Benzo[b]thiophen-4-yl)bicyclo[2.2.1]-hept-2-en-2-yl)methyl)dimethylamine

Into a solution of (+/−)-3-(N,N-dimethylamino)methyl-2-(2-trimethylsilylbenzo[b]thiophen-4-yl)bicyclo[2.2.1]heptan-2-ol, (0.2958 g, 0.791 mmol) in CH₂Cl₂ (7.9 mL) cooled at 0° C. was added TFA (3.1 mL), while stirring. After 2 h, TBAF (1.58 mL, 1.58 mmol, 1.0 M in THF) was added via an addition funnel. The mixture was stirred for 3 h, and then concentrated under reduced pressure. The oily residue was diluted with saturated aqueous NaHCO₃ (50 mL) and extracted with CH₂Cl₂ (3×50 mL). The combined organic layers were dried over MgSO₄, filtered and concentrated under reduced pressure. The residue was purified by silica gel chromatography (3% (2.0 M NH₃ in MeOH)/CH₂Cl₂). The purified fractions were concentrated under reduced pressure and placed under vacuum. The title compound turned to a white solid upon standing (0.2095 g, 93%). mp 73.5-76.9° C. IR (KBr) 2950, 2870, 2820, 2777, 1454 cm⁻¹. Ion Spray MS 284.1 (M^{+H)}⁺; 239.0 (M−N(CH₃)₂)⁻.

Similarly prepared were:

(+/−)-((3-(Benzo[b]thiophen-7-yl)bicyclo[2.2.1]-hept-2-en-2-yl)methyl)dimethylamine, mp 79.6-80.5° C. IR (KBr) 2949, 2870, 2820, 2777, 1456 cm⁻¹. Ion Spray MS 284.1 (M+H)⁺; 239.0 (M−N(CH₃)₂)⁺

(+/−)-Dimethyl((3-(2-methylbenzo[b]thiophen-5-yl)bicyclo[2.2.1]-hept-2-en-2-yl)methyl)amine, IR (KBr) 2962, 2950, 2921, 2870, 2819, 2775, 1446 cm⁻¹. FD+MS 297.3 (M)⁺

### EXAMPLE 16

(+/−)-Dimethyl((3-(2-methylbenzo[b]thiophen-5-yl)bicyclo[2.2.1]-hept-2-en-2-yl)methyl)amine hydrochloride

Into a solution of (+/−)-dimethyl((3-(2-methylbenzo [b]thiophen-5-yl)bicyclo[2.2.1]-hept-2-en-2-yl)methyl)amine,(0.26740 g, 0.898 mmol) in MeOH (1 mL) was added HCl (0.90 mL, 0.898 mmol, 1.0 M in Et₂O), while stirring. The mixture was stirred for 10 min, diluted with H₂O (20 mL), and lyophilized to afford an off-white powder, (0.2953 g, 98%). mp 206.5-214.2° C. Ion Spray MS 298.1 (M+H)⁺; 253.0 (M−N(CH₃)₂)⁺.

Similarly prepared were:

N,N-dimethyl [3-(2-methyl-1-benzothien-4-yl)bicyclo[2.2.1]hept-2-en-2-yl]methanamine hydrochloride, mp 181.6-189.1° C. IR (KBr) 2967, 1473 cm⁻¹. Ion Spray MS 298.1 (M+H)⁺; 253.0 (M−N(CH₃)₂)⁺

N,N-dimethyl[(1R,4S)-3-(2-methyl-1-benzothien-6-yl)bicyclo[2.2.1]hept-2-en-2-yl]methanamine hydrochloride, mp 68.2° C. (dec.). Ion Spray MS 298.3 (M+H)⁺; 253.2 (M−N(CH₃)₂)⁺

N,N-dimethyl [3-(2-methyl-1-benzothien-7-yl)bicyclo [2.2.1]hept-2-en-2-yl]methanamine hydrochloride, mp 179.5-181.6° C. Ion Spray MS 298.3 (M^{+H)}⁺; 253.2 (M−N(CH₃)₂)⁺

[3-(2-fluoro-1-benzothien-5-yl)bicyclo [2.2.1]hept-2-en-2-yl]-N,N-dimethylmethanamine, maleic acid, mp 168.1-168.8° C.

[3-(3-chloro-1-benzothien-5-yl)bicyclo[2.2.1]hept-2-en-2-yl]-N,N-dimethylmethanamine hydrochloride, mp 223-226° C.

N,N-dimethyl [3-(3-methyl-1-benzothien-5-yl) bicyclo [2.2.1]hept-2-en-2-yl]methanamine hydrochloride, FIA POS MS (M+H, 298.1)

N,N-dimethyl [3-(3-methyl-1-benzothien-7-yl) bicyclo [2.2.1]hept-2-en-2-yl]methanamine hydrochloride, mp 236-239° C.

N,N-dimethyl [3-(3-methyl-1-benzothien-6-yl) bicyclo [2.2.1]hept-2-en-2-yl]methanamine hydrochloride, mp 131-134° C.

N,N-dimethyl [3-(3-methyl-1-benzothien-4-yl)bicyclo [2.2.1]hept-2-en-2-yl]methanamine hydrochloride, mp 225° C.

[(1R,4S)-3-(1-benzothien-5-yl)bicyclo[2.2.1]hept-2-en-2-yl]-N,N-dimethylmethanamine hydrochloride, mp 236.4-236.7° C.

[(1S,4R)-3-(1-benzothien-5-yl)bicyclo[2.2.1]hept-2-en-2-yl]-N,N-dimethylmethanamine hydrochloride, mp 236.4-236.6° C.

[(1R, 4S) —N,N-dimethyl-3-(1-benzothien-4-yl)bicyclo[2.2.1]hept-2-en-2-yl]-methanamine hydrochloride, mp 254-257° C.,

[(1S,4R)-N,N-dimethyl-3-(1-benzothien-4-yl)bicyclo[2.2.1]hept-2-en-2-yl]-methanamine hydrochloride, mp 249-251° C.,

[N-methyl-(3-(3-methyl-1-benzothien-7-yl)bicyclo[2.2.1]hept-2-en-2-yl]-methanamine hydrochloride,

[(1R,4S)-N,N-dimethyl-3-(1-benzothien-7-yl)bicyclo[2.2.1]hept-2-en-2-yl]-methanamine hydrochloride,

[(1S,4R)-N,N-dimethyl-3-(1-benzothien-7-yl)bicyclo[2.2.1]hept-2-en-2-yl]-methanamine hydrochloride,

[N,N-dimethyl 3-(2-methoxymethyl-1-benzothien-6-yl)bicyclo[2.2.1]hept-2-en-2-yl]-methanamine hydrochloride, mp 184-187° C.,

[N,N-dimethyl 3-(2-hydroxymethyl-1-benzothien-6-yl)bicyclo[2.2.1]hept-2-en-2-yl]-methanamine hydrochloride, mp 183-186° C.,

[N,N-dimethyl 3-(2-{prop-2-enyl}-1-benzothien-6-yl)bicyclo[2.2.1]hept-2-en-2-yl]-methanamine hydrochloride, mp 160° C. (decomposed),

[N-methyl-3-(1-benzothien-4-yl)bicyclo [2.2.1]hept-2-en-2-yl]-methanamine hydrochloride, mp 216-218° C.,

[N-methyl-3-(6-fluoro-1-benzothien-3-yl)bicyclo[2.2.1]hept-2-en-2-yl]-methanamine hydrochloride, mp 237-240° C.,

[(1R,4S)-N-methyl-3-(1-benzothien-7-yl)bicyclo[2.2.1]hept-2-en-2-yl]-methanamine hydrochloride, mp 182-183° C.,

[(1S,4R)-N-methyl-3-(1-benzothien-7-yl)bicyclo[2.2.1]hept-2-en-2-yl]-methanamine hydrochloride, mp 181-183° C.,

[(1R,4S)-N,N-dimethyl-3-(3-methyl-1-benzothien-7-yl)bicyclo[2.2.1]hept-2-en-2-yl]-methanamine hydrochloride, mp 256° C.,

[(1S,4R)-N,N-dimethyl-3-(3-methyl-1-benzothien-7-yl)bicyclo[2.2.1]hept-2-en-2-yl]-methanamine hydrochloride, mp 256° C.,

[(1R,4S)-N,N-dimethyl-3-(1-benzothien-2-yl)bicyclo[2.2.1]hept-2-en-2-yl]-methanamine hydrochloride, mp 250-251° C.,

[(1S,4R)-N,N-dimethyl-3-(1-benzothien-2-yl)bicyclo[2.2.1]hept-2-en-2-yl]-methanamine hydrochloride, mp 251-252° C.,

[(1R,4S)-N-methyl-(3-(3-methyl-1-benzothien-7-yl)bicyclo[2.2.1]hept-2-en-2-yl]-methanamine hydrochloride, mp 196° C.,

[(1S,4R)-N-methyl-(3-(3-methyl-1-benzothien-7-yl)bicyclo[2.2.1]hept-2-en-2-yl]-methanamine hydrochloride, mp 247-249° C.,

[N-methyl-(3-(3-methyl-1-benzothien-6-yl)bicyclo [2.2.1]hept-2-en-2-yl]-methanamine hydrochloride, mp 205-206° C.,

[(1R,4S)-N-methyl-(3-(1-benzothien-7-yl)bicyclo[2.2.1]hept-2-en-2-yl]-methanamine hydrochloride, mp 229-233° C.,

[(1S,4R)-N-methyl-(3-(1-benzothien-7-yl)bicyclo [2.2.1]hept-2-en-2-yl]-methanamine hydrochloride, mp 224-227° C.,

[N-methyl-(3-(5-fluoro-1-benzothien-7-yl)bicyclo[2.2.1]hept-2-en-2-yl]-methanamine hydrochloride, mp 220° C.,

[N-methyl-(3-(3-cyano-1-benzothien-5-yl)bicyclo [2.2.1]hept-2-en-2-yl]-methanamine hydrochloride, mp 260-261° C.,

[N-methyl-(3-(1-benzofuran-5-yl)bicyclo[2.2.1]hept-2-en-2-yl]-methanamine hydrochloride, mp 78.8-79.4° C.,

[(1R,4S)-N-methyl-3-(6-fluoro-1-benzothien-3-yl)bicyclo [2.2.1]hept-2-en-2-yl]-methanamine hydrochloride, mp 250-252° C.,

[(1S,4R)-N-methyl-3-(6-fluoro-1-benzothien-3-yl) bicyclo [2.2.1]hept-2-en-2-yl]-methanamine hydrochloride, mp 251-253° C.,

### EXAMPLE 17

(+/−)-3-(N,N-Dimethylamino)methylbicyclo[3.2.1]octan-2-one

Into a slurry of bicyclo[3.2.1]octan-2-one (1.50 g, 12.07 mmol), dimethylamine hydrochloride (1.28 g, 15.70 mmol), and paraformaldehyde (0.478 g, 5.31 mmol) in EtOH (15 mL) was added concentrated HCl (0.4 mL), while stirring. The mixture was stirred at reflux for 3 days and concentrated under reduced pressure. Upon standing, crystals formed. The crystals were suspended in Et₂O (10 mL) and acetone (1 mL) for 15 min. The crystals were filtered and washed with Et₂O. The crystals were then dissolved in saturated aqueous NaHCO₃ (50 mL), forming the free base, and-extracted with Et₂O (3×50 mL). The combined organic layers were dried over MgSO₄, filtered and concentrated under reduced pressure. After being placed under vacuum, the title compound was obtained as a yellow oil, (1.1963 g, 54%). IR (KBr) 2947, 1703 cm⁻¹. Ion Spray MS 182.1 (M+H)⁺.

### EXAMPLE 18

3-{[allyl(methyl)amino]methyl}-bicyclo[3.2.1]octan-2-one

Bicyclo[3.2.1]octan-2-one (1.24 g, 10 mmol) was place in a 50 mL round bottomed flask equipped with a stirrer bar. A solution of dimethyl amine (10 mL, 20 mmol, 2.0 M solution in MeOH) was added followed by 40% aqueous formaldehyde solution (1.5 mL, 20 mmol) and c HCl (1 mL) The resulting solution was heated with stirring at 70° C. overnight. After cooling to room temperature the solvents were evaporated in vacuo. The residue was then partitioned between EtOAc and saturated aqueous Na₂CO₃ and the layers separated. The organic extract was washed with brine and dried over anh. MgSO₄, filtered and the solvent evaporated to give a brown oil (1.70 g, 9.4 mmol), FIA [M+1]=182.1. This oil was then taken up in MeOH (15 mL) and MeI (5 mL) added. Resulting solution was stirred at room temperature overnight. The solvents were evaporated in vacuo and the resulting residue taken up in DCM (25 mL) and 0.5% aqueous NaHCO₃ (25 mL) added. This solution was stirred at room temperature overnight. Layers separated and aqueous extract re-extracted with DCM. Organic extracts combined, dried over anh. MgSO₄₁ filtered and the solvent evaporated in vacuo to give a gum (1.07 g, 8.8 mmol). This gum was taken up in MeOH (10 mL) and N-methylallyl amine (910 μL, 9.5 mmol) added and stirred at room temperature overnight. The solvent was evaporated to give the desired product as a yellow oil (1.12 g, 5.4 mmol). Mass spec. positive FIA [M+H]=208.1.

### EXAMPLE 19

3-(1-pyrrolidinylmethyl)bicyclo[3.2.1]octan-2-one

3-(N,N-Dimethylamino)methylbicyclo[3.2.1]octan-2-one (2.0 g, 11 mmol) was taken up in MeOH (18 mL) and methyl iodide (6 mL) added with stirring. The reaction was stirred at room temperature overnight. Solvents evaporated and the residue re-suspended in DCM (30 mL) and 5% aqueous NaHCO₃ (30 mL) was added. The resulting solution was stirred at room temperature overnight. Layers separated and aqueous extract re-extracted with DCM. Organic extracts combined, dried over anh. MgSO₄, filtered and the solvent evaporated in vacuo to give a brown oil (1.38 g, 10.1 mmol). This oil taken up in MeOH (10 mL) and pyrrolidine (763 μL, 9.1 mmol) added. The resulting solution was stirred at room temperature overnight and the solvents evaporated in vacuo to the desired product as an oil (1.91 g, 9.22 mmol). Mass spec. positive FIA [M+H]=208.1.

Similarly prepared was:

3-(1-azetidinylmethyl)bicyclo[3.2.1]octan-2-one, Mass spec. positive FIA [M+H]=193.1.

### EXAMPLE 20

(+/−)-3-(N,N-Dimethylamino)methyl-2-(5-fluorobenzo[b]thiophen-2-yl)bicyclo[3.2.1]octan-2-ol

Into a stirred solution of 5-fluorobenzo[b]thiophene (0.209 mL, 1.379 mmol) and TMEDA (1.04 mL, 6.89 mmol) in THF (4 mL, freshly distilled) at −78° C. was added 1.6 M n-BuLi (0.95 mL, 1.51 mmol) via a syringe. After stirring for 40 min, a solution of (+/−)-3-(N,N-dimethylamino)methylbicyclo[3.2.1]octan-2-one (0.250 g, 1.379 mmol) in THF (2 mL) was added via a cannula. The solution was stirred overnight, while allowed to warm to room temperature, and concentrated under reduced pressure. The residue was diluted with saturated aqueous NaHCO₃ (50 mL). The mixture was extracted with CH₂Cl₂ (3×40 mL). The organic layers were combined, dried over MgSO₄, filtered and concentrated under reduced pressure. The residue was then purified by silica gel chromatography (3.5% (2.0 M NH₃ in MeOH)/CH₂Cl₂). The purified fractions were concentrated under reduced pressure and placed under vacuum, resulting in the title compound, (+/−)-3-(N,N-dimethylamino)methyl-2-(5-fluorobenzo[b]thiophen-2-yl)bicyclo[3.2.1]octan-2-ol as a white foam (0.2912 g, 63%). IR (KBr) 3004, 2952, 2863, 2831, 2787, 1443 cm⁻¹. Ion Spray MS 334.0 (M+H)⁺.

similarly prepared was:

3-{[allyl(methyl)amino]methyl}-2-[3-methyl-2-(trimethylsilyl)-1-benzothien-5-yl]bicyclo[3.2.1]octan-2-ol, Mass spec. positive FIA [M+H]=440.

### EXAMPLE 21

3-[(dimethylamino)methyl]-2-(6-fluoro-2-naphthyl)bicyclo [3.2.1]octan-2-ol

The enantiomers of 3-[(dimethylamino)methyl]-2-(6-fluoro-2-naphthyl)bicyclo[3.2.1]octan-2-ol were separated by chiral preparative chromatography, using a CHIRALPAK-AD column, and a mixture of Hexane/Ethanol 50/50 as the eluant FIA Pos MS (M+H, 328.1).

### EXAMPLE 22

(+/−)-dimethyl((2-(5-fluorobenzo[b]thiophen-2-yl)bicyclo[3.2.1]-oct-2-en-3-yl)methyl)amine hydrochloride

Into a solution of (+/−)-3-(N,N-dimethylamino)methyl-2-(5-fluorobenzo[b]thiophen-2-yl)bicyclo[3.2.1]octan-2-ol, (0.212 g, 0.637 mmol) in CH₂Cl₂ (7 mL) cooled at 0° C. was added TFA (2.5 mL), while stirring. The mixture was stirred for 3 days, and diluted with saturated aqueous NaHCO₃ (50 mL). The mixture was extracted with CH₂Cl₂ (3×50 mL). The combined organic layers were dried over MgSO₄, filtered and concentrated under reduced pressure. The residue was purified by silica gel chromatography (4% (2.0 M NH₃ in MeOH)/CH₂Cl₂). The purified fractions were concentrated under reduced pressure and placed under vacuum, resulting in (+/−)-Dimethyl((2-(5-fluorobenzo[b]thiophen-2-yl)bicyclo[3.2.1]-oct-2-en-3-yl)methyl)amine as a yellow oil,(0.1697 g, 84%). IR (KBr) 2943, 2864, 2824, 1604, 1444 cm⁻¹. Ion Spray MS 316.1 (M+H)⁺; 271.1 (M−N(CH₃)₂)⁺.

Into a solution of (+/−)-dimethyl((2-(5-fluorobenzo[b]thiophen-2-yl)bicyclo[3.2.1]-oct-2-en-3-yl)methyl)amine,(0.1346 g, 0.426 mmol) in MeOH (1 mL) was added HCl (0.42 mL, 0.426 mmol, 1.0 M in Et₂O), while stirring. The mixture was stirred for 10 min and diluted with H₂O (15 mL) and just enough CH₃CN to dissolve the salt (1 mL). The mixture was then lyophilized to afford a white powder,(0.1487 g, 99%). mp 197.9-203.8° C. Ion Spray MS 316.3 (M+H)⁺; 271.2 (M−N(CH₃)₂)⁺.

Similarly prepared were:

N,N-dimethyl[2-(2-naphthyl)bicyclo[3.2.1]oct-2-en-3-yl]methanamine, maleic acid, mp 164-166° C.

N,N-dimethyl[2-(6-fluoro-2-naphthyl)bicyclo[3.2.1]oct-2-en-3-yl]methanamine, maleic acid, mp 172-174° C.

N,N-dimethyl[(1S,5R)-2-(6-fluoro-2-naphthyl)bicyclo [3.2.1]oct-2-en-3-yl]methanamine, maleate, FIA Pos mass spec, (M+H 310.2)

N,N-dimethyl[(1R,5S)-2-(6-fluoro-2-naphthyl)bicyclo [3.2.1]oct-2-en-3-yl]methanamine, maleate, FIA Pos mass spec, (M+H 310.2/311.1)

[2-(7-fluoro-1-benzothien-2-yl)bicyclo[3.2.1]oct-2-en-3-yl]-N,N-dimethylmethanamine hydrochloride, mp 210.3-214.7° C. Ion Spray MS 316.3 (M+H)⁺; 271.1 (M−N(CH₃) 2)⁺

[2-(4-fluoro-1-benzothien-2-yl)bicyclo[3.2.1]oct-2-en-3-yl]-N,N-dimethylmethanamine hydrochloride, mp 216.1-218.4° C. Ion Spray MS 316.0 (M+H)⁺

[2-(6-fluoro-1-benzothien-2-yl)bicyclo[3.2.1]oct-2-en-3-yl]-N,N-dimethylmethanamine hydrochloride, mp 207.4-211.1° C. Ion Spray MS 316.0 (M+H)⁺; 270.9 (M−N(CH₃)₂)⁺

[(1S,5R)-2-(6-fluoro-1-benzothien-2-yl)bicyclo [3.2.1]oct-2-en-3-yl]-N,N-dimethylmethanamine hydrochloride, LC-MS (M+H, 316.1, 98.1%)

[(1R,5S)-2-(6-fluoro-1-benzothien-2-yl)bicyclo [3.2.1]oct-2-en-3-yl]-N,N-dimethylmethanamine hydrochloride, FIA POS MS (M+H, 316.1)

[2-(6-fluoro-1-benzothien-3-yl)bicyclo[3.2.1]oct-2-en-3-yl]-N,N-dimethylmethanamine hydrochloride, FIA POS MS (M+H, 316.2)

2-{3-[(dimethylamino)methyl]bicyclo[3.2.1]oct-2-en-2-yl}-1-benzothiophene-6-carbonitrile hydrochloride, FIA POS MS (M+H,323)

[2-(6-trifluoro-1-benzothien-3-yl)bicyclo[3.2.1]oct-2-en-3-yl]-N,N-dimethylmethanamine hydrochloride, mp 155-158° C.

N,N-dimethyl[3-(2-naphthyl)bicyclo[2.2.2]oct-2-en-2-yl]methanamine, maleate, mp 205° C.

[3-(6-methoxy-2-naphthyl)bicyclo[2.2.2]oct-2-en-2-yl]-N,N-dimethylmethanamine, maleate, mp 177° C.

[3-(1-benzothien-3-yl)bicyclo[2.2.2]oct-2-en-2-yl]-N,N-dimethylmethanamine, maleate mp 181° C.

6-(3-[(dimethylamino)methyl]bicyclo[2.2.2]oct-2-en-2-yl}-2-naphthol, maleate, mp 196° C.

[3-(6-fluoro-2-naphthyl)bicyclo[2.2.2]oct-2-en-2-yl]-N,N-dimethylmethanamine, maleate, mp 198° C.

[3-(6-fluoro-1-benzothien-3-yl)bicyclo[2.2.2]oct-2-en-2-yl]-N,N-dimethylmethanamine hydrochloride, FIA POS MS (M+H, 316.1)

1-{[3-(1-benzothien-5-yl)bicyclo[3.2.1]oct-2-en-2-yl]methyl}pyrrolidine hydrochloride, LC-MS pos m/z (M+H, 324, 100%), mp 135-138° C.

1-{[3-(1-benzothien-5-yl)bicyclo[3.2.1]oct-2-en-2-yl]methyl}azetidine hydrochloride, LC-MS pos m/z (M+H, 310, 100%), mp 97-99° C.

1-{[3-(3-methyl-1-benzothien-5-yl)bicyclo[2.2.2]oct-2-en-2-yl]methyl)pyrrolidine hydrochloride, FIA POS MS (M+H, 338.6)

[3-(1-benzothien-7-yl)bicyclo[2.2.2]oct-2-en-2-yl]-N-methylmethanamine hydrochloride, FIA POS MS (M+H, 284.1)

N-allyl-N-methyl-[3-(1-benzothien-5-yl)bicyclo[2.2.2]oct-2-en-2-yl]-methanamine hydrochloride, mp 218-221° C.,

N,N-dimethyl-[3-(3-methyl-1-benzothien-7-yl)bicyclo[2.2.2]oct-2-en-2-yl]-methanamine hydrochloride, mp 236-237° C.,

N,N-dimethyl-[3-(1-benzothien-2-yl)bicyclo[2.2.2]oct-2-en-2-yl]-methanamine hydrochloride, mp 226-228° C.,

### EXAMPLE 23

(+/−)-dimethyl[3-methyl-1-benzothien-5-yl]bicyclo[3.2.1]octan-2-ol

3-methyl-2-[3-methyl-2-(trimethylsilyl)-1-benzothien-5-yl]bicyclo[3.2.1]octan-2-ol (2.47 g, 6.2 mmol) was taken up in THF (20 mL) with stirring. A solution of TBAF (6.2 mL, 1M solution in THF with 5 wt. % water ex. Aldrich) was added and the resulting solution stirred at room temperature for 3 h. The solvents were evaporated and the residue taken up in ethyl acetate and washed with 5% NaHCO₃ solution and brine. After drying over MgSO₄ the solvent was removed in vacuuo to give an oil which was purified by column chromatography (hex:EtOAc, 8:2). Gave the desired product as an oil (1.12 g, 3.2 mmol). Mass spec. positive FIA [M+H]=330.2.

### EXAMPLE 24

[2-(1-Benzothien-5-yl)bicyclo[3.2.1]oct-2-en-3-yl]-N,N-dimethylmethanamine hydrochloride

[2-(1-benzothien-5-yl)-3-[(dimethylamino) methyl]bicyclo[3.2.1]octan-2-ol (1.0 g, 3.1 mmol) was treated with p-toluene sulfonic acid (2.67 g, 15 mmol, 5 equiv) in DCM (30 mL). The reaction was heated at reflux overnight. DCM was removed in vacuo and the residue taken up in ethyl acetate and washed successively with 1M NaOH solution (×5), brine, dried over anh. MgSO₄ and concentrated in vacuo to give an oil (880 mg, 2.83 mmol), Ion Spray MS 298.2 (M+H)⁺; 253.1 (M−N(CH₃)₂)⁺ Into a solution of [2-(1-benzothien-5-yl)bicyclo[3.2.1] oct-2-en-3-yl]-N,N-dimethylmethanamine (880 mg, 2.83 mmol) in MeOH (1 mL) was added HCl (0.29 mL, 2.83 mmol, 1.0 M in Et₂O), while stirring. The mixture was stirred for 10 min and diluted with H₂O (15 mL) and just enough CH₃CN to dissolve the salt (1 mL). The mixture was then lyophilized to afford a white powder, [2-(1-benzothien-5-yl)bicyclo[3.2.1]oct-2-en-3-yl]-N,N-dimethylmethanamine hydrochloride, mp 57.8° C.

Similarly prepared was:

N-allyl-N-methyl-N-([2-(3-methyl-1-benzothien-5-yl)bicyclo[3.2.1]oct-2-en-3-yl]methyl]amine, Mass spec. positive FIA [M+H]=338.3.

[(1R,5S)-2-(1-benzothien-5-yl)bicyclo[3.2.1]oct-2-en-3-yl]-N,N-dimethylmethanamine hydrochloride, mp 225.3-225.6° C.

[(1S,5R)-2-(1-benzothien-5-yl)bicyclo[3.2.1]oct-2-en-3-yl]-N,N-dimethylmethanamine hydrochloride, mp 225.6-226.0° C.

[2-(1-benzothien-6-yl)bicyclo[3.2.1]oct-2-en-3-yl]-N,N-dimethylmethanamine hydrochloride, mp 81.5° C. Ion Spray MS 298.1 (M+H)⁺; 253.0 (M−N(CH₃)₂)⁺

[2-(1-benzothien-4-yl)bicyclo[3.2.1]oct-2-en-3-yl]-N,N-dimethylmethanamine hydrochloride, mp 182.4° C. Ion Spray MS 298.2 (M+H)⁺; 253.1 (M−N(CH₃)₂)⁺

[(1S,5R)-2-(1-benzothien-4-yl)bicyclo[3.2.1]oct-2-en-3-yl]-N,N-dimethylmethanamine hydrochloride, LC-MS (M+H, 298.1, 100%)

[(1R,5S)-2-(1-benzothien-4-yl)bicyclo[3.2.1]oct-2-en-3-yl]-N,N-dimethylmethanamine hydrochloride, LC-MS (M+H, 298.1, 100%)

[2-(1-benzothien-7-yl)bicyclo[3.2.1]oct-2-en-3-yl]-N,N-dimethylmethanamine hydrochloride, mp 205.0° C. (dec.). Ion Spray MS 298.1 (M+H)⁺; 253.1 (M−N(CH₃)₂)⁺

[(1S,5R)-2-(1-benzothien-7-yl)bicyclo[3.2.1]oct-2-en-3-yl]-N,N-dimethylmethanamine hydrochloride, mp 244-245° C.

[(1R,5S)-2-(1-benzothien-7-yl)bicyclo[3.2.1]oct-2-en-3-yl]-N,N-dimethylmethanamine hydrochloride, mp 245-246° C.

[2-(3-chloro-1-benzothien-5-yl)bicyclo[3.2.1]oct-2-en-3-yl]-N,N-dimethylmethanamine hydrochloride, mp 233-235° C.

N,N-dimethyl[-2-(3-methyl-1-benzothien-5-yl)bicyclo[3.2.1]oct-2-en-3-yl]methanamine hydrochloride, FIA POS MS (M+H, 312.1)

N,N-dimethyl[(1S,5R)-2-(3-methyl-1-benzothien-5-yl)bicyclo[3.2.1]oct-2-en-3-yl]methanamine hydrochloride, FIA POS MS (M+H, 312.1)

N,N-dimethyl[(1R,5S)-2-(3-methyl-1-benzothien-5-yl)bicyclo[3.2.1]oct-2-en-3-yl]methanamine hydrochloride, FIA POS MS (M+H, 312.1)

N,N-dimethyl[2-(3-methyl-1-benzothien-7-yl)bicyclo [3.2.1]oct-2-en-3-yl]methanamine hydrochloride,

[3-(1-benzothien-5-yl)bicyclo[2.2.2]oct-2-en-2-yl]-N,N-dimethylmethanamine hydrochloride, mp 209.7-210.0° C.

[3-(1-benzothien-6-yl)bicyclo[2.2.2]oct-2-en-2-yl]-N,N-dimethylmethanamine hydrochloride, mp 206.4-206.9° C.

[3-(3-chloro-1-benzothien-5-yl)bicyclo[2.2.2]oct-2-en-2-yl]-N,N-dimethylmethanamine hydrochloride, mp 224.6-225.9° C.

N,N-dimethyl[3-(3-methyl-1-benzothien-5-yl)bicyclo [2.2.2]oct-2-en-2-yl]methanamine hydrochloride, FIA POS MS (M+H, 312.1)

[3-(1-benzothien-7-yl)bicyclo[2.2.2]oct-2-en-2-yl]-N,N-dimethylmethanamine hydrochloride, mp 254-256° C.

[3-(1-benzothien-4-yl)bicyclo[2.2.2]oct-2-en-2-yl]-N,N-dimethylmethanamine hydrochloride, mp 207.8-209.9° C.

N,N-dimethyl-[(1S,5R)-2-(3-chloro-1-benzothien-5-yl)bicyclo[3.2.1]oct-2-en-3-yl]methanamine hydrochloride,

N,N-dimethyl-[(1R,5S)-2-(3-chloro-1-benzothien-5-yl)bicyclo[3.2.1]oct-2-en-3-yl]methanamine hydrochloride,

N,N-dimethyl-[(1S,5R)-2-(6-fluoro-1-benzothien-3-yl)bicyclo[3.2.1]oct-2-en-3-yl]methanamine hydrochloride, mp 248-249° C.,

N,N-dimethyl-[(1R,5S)-2-(6-fluoro-1-benzothien-3-yl)bicyclo[3.2.1]oct-2-en-3-yl]methanamine hydrochloride, mp 248-249° C.,

N,N-dimethyl-[(1S,5R)-2-(3-methyl-1-benzothien-7-yl)bicyclo[3.2.1]oct-2-en-3-yl]methanamine hydrochloride, mp 254° C.,

N,N-dimethyl-[(1S,5R)-2-(3-methyl-1-benzothien-7-yl)bicyclo[3.2.1]oct-2-en-3-yl]methanamine hydrochloride, mp 256-258° C.,

N,N-dimethyl-[(1S,5R)-2-(6-fluoro-naphth-2-yl)bicyclo[3.2.1]oct-2-en-3-yl]methanamine hydrochloride, mp 207-208° C.,

N,N-dimethyl-[(1R,5S)-2-(6-fluoro-naphth-2-yl)bicyclo[3.2.1]oct-2-en-3-yl]methanamine hydrochloride, mp 214-215° C.,

### EXAMPLE 25

N-Methyl-N-{[2-(3-methyl-1-benzothien-5-yl)bicyclo[3.2.1]oct-2-en-3-yl]methyl}amine, Hydrochloride Salt,

N-allyl-N-methyl-N-{[2-(3-methyl-1-benzothien-5-yl)bicyclo[3.2.1]oct-2-en-3-yl]methyl}amine (880 mg, 2.61 mmol) was taken up in dry, degassed DCM (10 mL) under N₂ atmosphere. This solution was transferred via a cannular into a second flask containing Pd(PPh₃)₄ (59 mg, 0.052 mmol) and 1,3-dimethylbarbituric acid (1.22 g, 7.83 mmol). The resulting solution was stirred at 40° C. for 5 h. Reaction cooled to room temperature and diluted with DCM. The resulting solution was washed with saturated Na₂CO₃ solution (×5), dried over anh. MgSO₄ and concentrated in vacuo to give an oil which was purified by column chromatography (MeOH:DCM, 1:9 an 1% of 2M NH₃/MeOH solution) to give a gum (360 mg, 1.21 mmol). This was converted to the HCl salt using 1.0M solution HCl in ether to give the desired product as a white solid. MP=204.5-206.7 and mass spec. positive FIA [M+H]=298.2

Similarly prepared was:

[3-(6-fluoro-2-naphthyl)bicyclo[3.2.1]oct-2-en-2-yl]-N-methylmethanamine, hydrochloride salt, mp 200.4-202.8° C.,

N-methyl-[3-(3-methyl-1-benzothien-7-yl)bicyclo[2.2.2]oct-2-en-2-yl]-methanamine hydrochloride, mp 200° C. (decomposed),

N-methyl-[3-(6-fluoro-1-benzothien-3-yl)bicyclo[2.2.2]oct-2-en-2-yl]-methanamine hydrochloride, mp 269-270° C.,

N-methyl-[3-(1-benzothien-4-yl)bicyclo[2.2.2]oct-2-en-2-yl]-methanamine hydrochloride,

N-methyl-[3-(3-methyl-1-benzothien-5-yl)bicyclo[2.2.2]oct-2-en-2-yl]-methanamine hydrochloride, mp 229-233° C.,

N-methyl-[3-(1-benzothien-5-yl)bicyclo[2.2.2]oct-2-en-2-yl]-methanamine hydrochloride, mp 218-221° C.,

N-methyl-[2-(3-methyl-1-benzothien-5-yl)bicyclo[3.2.1]oct-2-en-3-yl]methanamine hydrochloride, mp 204.5-206.7° C.,

N-methyl-[2-(1-benzothien-7-yl)bicyclo[3.2.1]oct-2-en-3-yl]methanamine hydrochloride, mp 212-213° C.,

N-methyl-[2-(3-methyl-1-benzothien-7-yl)bicyclo [3.2.1]oct-2-en-3-yl]methanamine hydrochloride, mp 130° C.,

N-methyl-[2-(6-fluoro-1-benzothien-3-yl)bicyclo[3.2.1]oct-2-en-3-yl]methanamine hydrochloride, mp 244-246° C.,

N-methyl-[(1S, 5R)-2-(6-fluoro-naphth-2-yl)bicyclo[3.2.1]oct-2-en-3-yl]methanamine hydrochloride, mp 219-220° C.,

N-methyl-[(1R, 5S)-2-(6-fluoro-naphth-2-yl)bicyclo[3.2.1]oct-2-en-3-yl]methanamine hydrochloride, mp 223-224° C.,

N-methyl-[2-(1-benzothien-4-yl)bicyclo[3.2.1]oct-2-en-3-yl]methanamine hydrochloride, mp 231-233° C.,

N-methyl-[(1S,2R)-2-(6-cyano-1-benzothien-2-yl)bicyclo[3.2.1]oct-2-en-3-yl]methanamine hydrochloride,

N-methyl-[(1R,2S)-2-(6-cyano-1-benzothien-2-yl)bicyclo[3.2.1]oct-2-en-3-yl]methanamine hydrochloride,

N-methyl-[(1S,2R)-2-(1-benzothien-7-yl) bicyclo [3.2.1]oct-2-en-3-yl]methanamine hydrochloride, mp 224-227° C.,

N-methyl-[(1R,2S)-2-(1-benzothien-7-yl)bicyclo[3.2.1]oct-2-en-3-yl]methanamine hydrochloride, mp 226-227° C.,

N-methyl-[(1S,2R)-3-(3-methyl-1-benzothien-5-yl)bicyclo[2.2.2]oct-2-en-2-yl]-methanamine hydrochloride, mp 200-202° C.,

N-methyl-[(1R,2S)-3-(3-methyl-1-benzothien-5-yl)bicyclo [2.2.2]oct-2-en-2-yl]-methanamine hydrochloride, mp 204-205° C.,

N-methyl-[(1S,2R)-2-(1-benzothien-4-yl)bicyclo[3.2.1]oct-2-en-3-yl]methanamine hydrochloride, mp 235-237° C.,

N-methyl-[(1R,2S)-2-(1-benzothien-4-yl) bicyclo [3.2.11 oct-2-en-3-yl]methanamine hydrochloride, mp 235-237° C.,

N-methyl-2-(3-cyano-1-benzothien-5-yl)bicyclo[3.2.1]oct-2-en-3-yl]methanamine hydrochloride, mp 226-227° C.,

N-methyl-[2-(1-benzothien-2-yl)bicyclo[3.2.1]oct-2-en-3-yl]methanamine hydrochloride, mp 215-216° C.,

N-methyl-[2-(5-methyl-1-benzothien-2-yl)bicyclo [3.2.1]oct-2-en-3-yl]methanamine hydrochloride, mp 169-170° C.,

N-methyl-[2-(6-fluoro-1-benzothien-2-yl)bicyclo[3.2.1]oct-2-en-3-yl]methanamine hydrochloride, mp 173-176° C.,

N-methyl-[2-(7-fluoro-1-benzothien-4-yl)bicyclo[3.2.1]oct-2-en-3-yl]methanamine hydrochloride, mp 234-236° C.,

N-methyl-[2-(3-methyl-1-benzothien-4-yl)bicyclo[3.2.1]oct-2-en-3-yl]methanamine hydrochloride,

N-methyl-[2-(5-methylthio-1-benzothien-2-yl)bicyclo[3.2.1]oct-2-en-3-yl]methanamine hydrochloride, mp 169-171° C.,

N-methyl-[2-(1-benzofuran-7-yl)bicyclo[3.2.1]oct-2-en-3-yl]methanamine hydrochloride, mp 249-251° C.,

N-methyl-[2-(1-benzofuran-6-yl)bicyclo[3.2.1]oct-2-en-3-yl]methanamine hydrochloride, mp 278.2-279.1° C.,

### EXAMPLE 26

N,N-Dimethyl [3-(6-{[4-(trifluoromethyl)benzyl]oxy}-2-naphthyl)bicyclo[2.2.2]oct-2-en-2-yl]methanamine,

A 100 ml round bottomed flask was charged with a 0.06M solution of 7-{3-[(dimethylamino)methyl]-bicyclo[2.2.2]oct-2-en-2-yl)-2-naphthol (220 mg, 0.716 mmol) in amine-free N,N-dimethylformamide (DMF, 11.92 mls). To this solution was added a solution of sodium bis(trimethylsilyl)amide (1M in tetrahydrofuran, 1.073 ml, 1.073 mmol, 1.5 eq). The flask was flushed with nitrogen, stoppered and its contents stirred at r.t. for one hour, during which time the solution changed in colour from yellow to dark brown. Twenty alkyl and benzyl halides were each dissolved in amine-free DMF (550 μl to 100 μl) such that in each case, a 0.12M solution was made up. An aliquot of each (500 μl, 0.06 mmol) was placed in its own 4 ml Reacti-Vial. To each Vial, under a blanket of nitrogen, was added an aliquot of the naphtholate solution prepared above (500 μl, 0.03 mmol). The twenty Vials were sealed, and their contents stirred and heated to 80° C. overnight. The Vials were then unsealed and in each case, the contents were treated with methanol (1 ml). Each mixture was passed through its own methanol-conditioned 500 mg SCX ion-exchange cartridge (under gravity) into a collection tank. Each cartridge was then washed with fresh methanol (2×2.5 ml) such that the washings also passed into the tank. A clear glass vial was then placed underneath each cartridge, which were then eluted with 2M ammonia in methanol to release the products. Solvents were removed in vacuo to recover the target materials. FIA POS MASS SPEC (M+H, 466 & [M−N(CH₃)₂]⁺, 421).

Similarly prepared were:

[3-(6-methoxy-2-naphthyl)bicyclo[2.2.2]oct-2-en-2-yl]-N,N-dimethylmethanamine, FIA POS MASS SPEC (M+H, 322 & [M−N(CH₃)₂]⁺, 277)

[3-(6-butoxy-2-naphthyl)bicyclo[2.2.2]oct-2-en-2-yl]-N,N-dimethylmethanamine, FIA POS MASS SPEC (M+H, 364 & [M−N(CH₃)₂]⁺, 319)

(3-[6-(benzyloxy)-2-naphthyl]bicyclo[2.2.2]oct-2-en-2-yl}-N,N-dimethylmethanamine, FIA POS MASS SPEC (M+H, 398 & [M−N(CH₃)₂]⁺, 353)

(3-{6-[(4-bromobenzyl)oxy]-2-naphthyl}bicyclo[2.2.2]oct-2-en-2-yl)-N,N-dimethylmethanamine, FIA POS MASS SPEC (M+H, 475/477 & [M−N(CH₃)₂]⁺, 430/432)

4-{[(6-[3-[(dimethylamino)methyl]bicyclo[2.2.2]oct-2-en-2-yl}-2-naphthyl)oxy]methyl}benzonitrile, FIA POS MASS SPEC (M+H, 423 & [M−N(CH₃)₂]⁺, 378)

(3-{6-[(3-chlorobenzyl)oxy]-2-naphthyl}bicyclo[2.2.2]oct-2-en-2-yl)-N,N-dimethylmethanamine, FIA POS MASS SPEC (M+H, 432 & [M−N(CH₃)₂]⁺, 387)

N,N-dimethyl(3-{6-[(4-methylbenzyl)oxy]-2-naphthyl}bicyclo[2.2.2]oct-2-en-2-yl)methanamine, FIA POS MASS SPEC (M+H, 412 & [M−N(CH₃)₂]⁺, 367)

(3-{6-[(2,6-dichlorobenzyl)oxy]-2-naphthyl}bicyclo [2.2.2]oct-2-en-2-yl)-N,N-dimethylmethanamine, FIA POS MASS SPEC (M+H, 466/468 & [M−N(CH₃)₂]⁺, 421/423)

[3-(6-ethoxy-2-naphthyl)bicyclo[2.2.2]oct-2-en-2-yl]-N,N-dimethylmethanamine, FIA POS MASS SPEC (M+H, 336 & [M−N(CH₃)₂]⁺, 291)

(3-{6-[(4-tert-butylbenzyl)oxy]-2-naphthyl}bicyclo [2.2.2]oct-2-en-2-yl)-N,N-dimethylmethanamine, FIA POS MASS SPEC (M+H, 454& IM-N(CH₃)₂]⁺, 409)

(3-{6-[(2-chlorobenzyl)oxy]-2-naphthyl}bicyclo [2.2.2]oct-2-en-2-yl)-N,N-dimethylmethanamine, FIA POS MASS SPEC (M+H, 432 & [M−N(CH₃)₂]⁺, 387)

(3-{6-[(4-fluorobenzyl)oxy]-2-naphthyl}bicyclo [2.2.2]oct-2-en-2-yl)-N,N-dimethylmethanamine, FIA POS MASS SPEC (M+H, 416 & [M−N(CH₃)₂]⁺, 371)

N,N-dimethyl[3-(6-propoxy-2-naphthyl)bicyclo[2.2.2]oct-2-en-2-yl]methanamine, FIA POS MASS SPEC (M+H, 350 & [M−N(CH₃)₂]⁺, 305)

(3-{6-[(4-methoxybenzyl)oxy]-2-naphthyl}bicyclo [2.2.2]oct-2-en-2-yl)-N,N-dimethylmethanamine, FIA POS MASS SPEC (M+H, 428 & [M−N(CH₃)₂]⁺, 383)

N,N-dimethyl(3-{6-[(4-nitrobenzyl)oxy]-2-naphthyl}bicyclo[2.2.2]oct-2-en-2-yl)methanamine, FIA POS MASS SPEC (M+H, 443 & [M−N(CH₃)₂]⁺, 398)

N,N-dimethyl[3-(6-{[4-(trifluoromethoxy)benzyl]oxy}-2-naphthyl)bicyclo[2.2.2]oct-2-en-2-yl]methanamine, FIA POS MASS SPEC (M+H, 482 & [M−N(CH₃)₂]⁺, 437)

### EXAMPLE 27

Tablets each containing 10 mg of active ingredient are made up as follows:

| | |
|---|---|
| Active ingredient | 10 mg |
| Starch | 160 mg |
| Microcrystalline cellulose | 100 mg |
| Polyvinylpyrrolidone (as 10% solution in water) | 13 mg |
| Sodium carboxymethyl starch | 14 mg |
| Magnesium stearate | 3 mg |
| Total | 300 mg |

The active ingredient, starch and cellulose are mixed thoroughly. The solution of polyvinylpyrrolidone is mixed with the resultant powders and passed through a sieve. The granules so produced are dried and re-passed through a sieve. The sodium carboxymethyl starch and magnesium stearate are then added to the granules which, after mixing, are compressed on a tablet machine to yield tablets each weighing 300 mg.

### EXAMPLE 28

Capsules each containing 20 mg of medicament are made as follows:

| | |
|---|---|
| Active ingredient | 20 mg |
| Dried starch | 178 mg |
| Magnesium stearate | 2 mg |
| Total | 200 mg |

The active ingredient, starch and magnesium stearate are passed through a sieve and filled into hard gelatine capsules in 200 mg quantities.

### EXAMPLE 29

[³H]-Citalopram Binding Assay

The ability of compounds of the invention to displace [³H]-citalopram from binding sites on rat cerebral cortical membranes was measured in the following way:

In each well of a 96 deep well plate was added:

| | |
|---|---|
| 100 ml | 2 nM [³H] - citalopram |
| 600 ml | 50 mM Tris.HCl pH 7.4 containing 150 mM NaCl and |
| | 5 mM KCl |
| 100 ml | Diluted compound, 50 mM Tris.HCl pH 7.4 |
| | containing 150 mM NaCl and 5 mM KCl (total |
| | binding) or 100 mM fluoxetine (non-specific |
| | binding) |
| 200 ml | Membrane preparation (0.75 mg protein per ml) |

The microtitre plates were incubated at 37□C for 90 minutes followed by filtration through GF/B filters soaked in 50 mM Tris.HCl/0.1% (w/v) polyethylenimine pH 7.4. The filter was washed 5 times with 50 mM Tris.HCl. pH 7.4. The filters were removed, dried and the bound tritium determined by liquid scintillation spectrometry. The results were analysed using an automatic spline fitting programme to provide Ki values for each of the compounds.

1. A compound of the formula 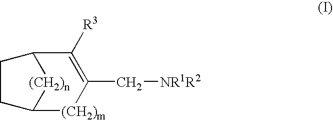
in which R¹ and R² are each hydrogen or C₁₋₄ alkyl, or R¹ and R² together with the nitrogen atom to which they are attached form an azetidinyl, pyrrolidinyl, piperidinyl, piperazinyl or morpholino group, said group being optionally substituted with 1 to 3 substituents selected from C₁₋₄ alkyl, hydroxymethyl, C₁₋₄ alkoxymethyl and amido,
R³ is a naphthyl, indolyl, benzothienyl, benzofuranyl, benzothiazolyl, quinolinyl or isoquinolinyl group, said group being optionally substituted with 1 to 3 substituents selected from C₁₋₄ alkyl, C₁₋₄ alkoxy, carboxy, nitro, hydroxy, cyano, halo, trifluoromethyl, trifluoromethoxy, optionally substituted benzyl, optionally substituted benzyloxy, —NR′R″, —CONR′R″, —SO₂NR′R″ and —SO₂R′, where R′ and R″ are each hydrogen or C₁₋₄ alkyl;
n is 1 or 2 and m is 0 or 1, provided that when m is 1 then n is 1;
or a salt or ester thereof.

2. A compound according to claim 1, in which R¹ and R² are each hydrogen or C₁₋₄ alkyl.

3. A compound according to either of claims 1 and 2, in which R³ is optionally substituted 2-, 3-, 4-, 5-, 6- or 7-benzothienyl.

4. A compound according to claims 3, in which R³ is optionally substituted 4-,5- or 7-benzothienyl.

5. A compound according to claim 1, of the formula 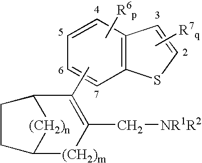
in which R¹ and R² are each hydrogen or C₁₋₄ alkyl, R⁶ and R⁷ are each C₁₋₄ alkyl, C₁₋₄ alkoxy, carboxy, hydroxy, cyano, halo, trifluoromethyl, —NR′R″, —CONR′R″, —SO₂NR′R″ or —SO₂R¹ where R′ and R″ are each hydrogen or C₁₋₄ alkyl, and p and q are each 0, 1 or 2; or a salt thereof.

6. A pharmaceutical formulation comprising a compound according to any of claims 1 to 5, or pharmaceutically acceptable salt or ester thereof, and a pharmaceutically acceptable diluent or carrier therefor.

7. A compound according to any of claims 1 to 5, or a pharmaceutically acceptable salt or ester thereof, for use as a pharmaceutical.

8. A compound according to any of claims 1 to 5, or a pharmaceutically acceptable salt or ester thereof, for treating a disorder of the central nervous system.

9. The use of a compound according to any of claims 1 to 5, or a pharmaceutically acceptable salt or ester thereof; for the manufacture of a medicament for treating a disorder of the central nervous system.

10. A method of treating an animal, including a human, suffering or susceptible to a disorder of the central nervous system, which comprises administering a compound according to claim 1, or a pharmaceutically acceptable salt or ester thereof.
